(19) United States
(12) Patent Application Publication
(10) Pub. No.: US 2011/0195420 A1
(43) Pub. Date: Aug. 11, 2011

(54) ESTROGEN AND ANTI-ESTROGEN MARKER GENES

(75) Inventors: Stefan Mueller, Bensheim (DE); Kathleen Boehme, Leipzig (DE)

(73) Assignee: Merck Patent Gesellschaft, Darmstadt (DE)

(21) Appl. No.: 13/122,987

(22) PCT Filed: Sep. 18, 2009
(86) PCT No.: PCT/EP09/06749

§ 371 Date: Apr. 7, 2011

(51) Int. Cl. C12Q 1/68

(52) U.S. Cl. 435/6.13

(57) ABSTRACT

The invention relates to a method for screening compounds with estrogenic or anti-estrogenic activity by providing a cellular system of a sample thereof being capable of expressing at least a single gene of Table 1, incubating at least a portion of the system with compounds to be screened, and comparing an expression of the single gene of Table 1 in the system with the gene expression in a control cellular system. Another object of the invention concerns a method for monitoring physiological and/or pathological conditions, which are caused, mediated and/or propagated by estrogen receptor signaling, by administering an effective amount of at least a single compound to a mammal in need of such treatment and determining an expression of the single gene of Table 1 in a biological sample withdrawn from the mammal. The invention also relates to the use of the genes of Table 1 as well as substances specifically interacting with gene products encoded by the genes of Table 1.

The invention relates to a method for screening compounds with estrogenic or anti-estrogenic activity by providing a cellular system of a sample thereof being capable of expressing at least a single gene of Table 1, incubating at least a portion of the system with compounds to be screened, and comparing an expression of the single gene of Table 1 in the system with the gene expression in a control cellular system. Another object of the invention concerns a method for monitoring physiological and/or pathological conditions, which are caused, mediated and/or propagated by estrogen receptor signaling, by administering an effective amount of at least a single compound to a mammal in need of such treatment and determining an expression of the single gene of Table 1 in a biological sample withdrawn from the mammal. The invention also relates to the use of the genes of Table 1 as well as substances specifically interacting with gene products encoded by the genes of Table 1.

An endocrine active compound (EAC) is an exogenous agent of anthropogenous and natural origin that can interfere with or mimic the physiologic function of endogenous hormones by altering hormone synthesis, secretion, transport, binding, action or elimination. EACs may either cause a variety of adverse health effects, which are related to development, reproduction, nervous and immune systems in human and wildlife populations, or have beneficial effects on estrogen-dependent diseases, warranting a thorough analysis regarding their mode of action.

Arising from different chemical classes with differing structures, it is not surprising that EAC effects on the endocrine system are mediated by a variety of mechanisms. A major mechanism involves steroid hormone receptors that belong to the nuclear receptor superfamily and regulate gene transcription. Estrogen receptor ERα and ERβ are well-described targets of estrogenic chemicals, including many everyday life products such as pharmaceuticals, pesticides or food ingredients. Although all compounds share a similarity to mediate ER-dependent gene transcription, in the public mind, phytoestrogens, a large family of naturally occurring plant-derived compounds, are mostly considered as beneficial, whereas synthetic EACs are associated with harmful effects. However, scientific investigations do not support this generalized view. Experimental outcomes are dependent on multiple factors, such as the treatment concentration, used species, tissue or cell type, and the intrinsic estrogen status. Only a deeper understanding of mechanisms underlying the biological effects can help to understand this controversy and support the assessment of relevance for humans and the environment.

Consequently, endocrine disruption has become an important issue, as many environmental compounds of differing provenance and chemical class may induce hormonal imbalances in many species by binding to the estrogen receptors and stimulating or inhibiting estrogen responsive genes. This may be followed by changing expression levels of many other genes, controlling several essential cellular processes. However, rapid non-genomic effects involving the steroid-induced modulation of mitogen-activated protein kinase, phosphatidylinositol 3-OH kinase, GPCR and growth factor signaling cascades can also cause alterations in the transcriptome. Thus knowledge about variations in global gene expression is more important for reliable evaluation of estrogenicity than investigating initial binding events to the ER.

Many of the putative marker genes for estrogenicity found for DES were regulated to a lesser extent by Genistein (GEN), Zearalenone (ZEA), Bisphenol A (BPA), o,p′-Dichlordiphenyltrichlorethane (DDT) and Resveratrol (RESV), which confirms the lower estrogenic potencies reported for these compounds (Mueller et al. 2004 Toxicol Sci 80(1): 14-25). Such lower potencies complicate risk assessment and extrapolation of low dose effects from animal studies to humans, indicating the need of a more sensitive experimental system for the evaluation of estrogenicity and estimation of dose-response-relationships. In particular, results for BPA and DDT give cause for thought, because even at doses 50 to 100 times higher than GEN and ZEA, the marker gene regulations were often weaker than for the phytoestrogens.

The importance for new screening tools is also illustrated by REACH (Registration, Evaluation, Authorization and Restriction of Chemicals), the new European Community regulation on chemicals (European Commission 2007). In accordance with REACH, around 30,000 existing chemicals have to be tested and endocrine disruption is one essential issue within the REACH testing program. However, enormous costs and animal welfare issues make it difficult to fulfill REACH requirements using existing, traditional toxicological assays.

It has been recently suggested in US 2003/0008309 A1 that the effects of environmental hormones can be detected by evaluating chemical substances having estrogen-like activity. DNA fragments containing portions or wholes of genes and/or ESTs (Expressed Sequence Tags) whose expression is affected by chemical substances having estrogen-like activity are immobilized on the basal plate of the microarray. However, prior art is mainly restricted to expression level changes in the breast cancer-derived MCF-7 cell and lacks the compilation of distinct expression profiles.

U.S. Pat. No. 7,371,207 B2 teaches a plurality of genes, each of whom is differentially expressed in kidney cells exposed to estrogen and/or other hormones or combination of hormones and kidney cells without said exposure, which plurality comprises a first group and a second group, wherein each gene in said first group is differentially expressed at a higher level in said kidney cells exposed to estrogen and/or other hormones or combination of hormones than in said kidney cells without said exposure, and wherein each gene in said second group is differentially expressed at a lower level in said kidney cells exposed to estrogen and/or other hormones or combination of hormones than in said kidney cells without said exposure. However, the genes are limited such that said first group comprises the full-length genes NTT73, CYP7B 1 and ABCC3, and said second group comprises the full-length genes BHMT and SAHH, which reduces screening stability, but enhances error rates.

Therefore, the technical problem forming the basis of the present invention is to provide a method for screening compounds, which effectively allows the identification and characterization of their estrogenic or anti-estrogenic properties. It is another problem of the invention to provide substances for the detection of estrogenic or anti-estrogenic activity, which makes a simple and fast monitoring of estrogen-dependent diseases possible.

The present invention solves the problem by providing a method for screening compounds with estrogenic or anti-estrogenic activity comprising the steps of:
(a) providing a cellular system or a sample thereof being capable of expressing at least one gene of Table 1, wherein the system is selected from the group of single cells, cell cultures, tissues, organs and mammals,
(b) incubating at least a portion of the system with compounds to be screened, and
(c) detecting the activity by comparing an expression of the at least one gene of Table 1 in the system with the gene expression in a control cellular system.

It has been surprisingly demonstrated by the inventors that the aforementioned group of 72 genes is correlated with estrogenicity. Consequently, the aforementioned plurality of marker genes represent novel estrogen/estrogen receptor target genes, which themselves and their gene products, respectively, are well suited targets for differentiating the stage of estrogenicity. The underlying genes are selected as result of a differential expression analysis. The identified genes are not inevitably associated by function or location in their entity as presently known, but it is not excluded that such relations appear between a single member or more members of the group. Instead of that, all genes are characterized by a distinct difference to EAC-untreated cells, which is exhibited by either up-regulation or repression. The genes are already described in the state of the art by sequence and other features, but lacking a linkage to estrogenicity. The similar gene regulation reflects the ability of EACs to exert estrogen-like growth stimulatory activity through the up-regulation of proliferation promoting genes and down-regulation of negative proliferation regulators and apoptosis inducing genes. The aforementioned genes may be named in another way, but are easily assigned by the accession number, which is generally accepted and fixed in numerous data bases, such as the GenBank, SwissProt and the like.

The linkage of estrogenicity to distinct genes is utilized for the in-vitro detection of endocrine active compounds, which are able to interfere with estrogen receptor signaling, and anti-estrogenic compounds. Building a compound specific gene expression profile, which is based on the plurality of genes according to Table 1, is of unexpected benefit in establishing an estrogenic or anti-estrogenic mechanism of action and, therefore, supports the evaluation of potential hazards or benefits of novel compounds supplementary to the classical screening methods. Either a single marker gene or more than a single marker can be used for the utmost test reliability. That means the inventive principle underlying the present method comprises prospecting for a gene product that can be either detected on the genetic level or on the protein level, wherein the genetic level is preferred. The gene product is chosen in respect of both its absolute and relative amount as well as the specificity for a certain cell type.

In general, “a gene” is a region on the genome that is capable of being transcribed to RNA that either has a regulatory function, a catalytic function and/or encodes a protein. A gene typically has introns and exons, which may organize to produce different RNA splice variants that encode alternative versions of a mature protein. “Gene” contemplates fragments of genes that may or may not represent a functional domain.

A “plurality of genes” as used herein refers to a group of identified or isolated genes whose levels of expression vary in different tissues, cells or under different conditions or biological states. The different conditions may be caused by exposure to certain agent(s)—whether exogenous or endogenous—which include hormones, receptor ligands, chemical compounds and the like. The expression of a plurality of genes demonstrates certain patterns. That is, each gene in the plurality is expressed differently in different conditions or with or without exposure to a certain endogenous or exogenous agents. The extent or level of differential expression of each gene may vary in the plurality and may be determined qualitatively and/or quantitatively according to this invention. A gene expression profile, as used herein, refers to a plurality of genes that are differentially expressed at different levels, which constitutes a “pattern” or a “profile.” As used herein, the term “expression profile”, “profile”, “expression pattern”, “pattern”, “gene expression profile” and “gene expression pattern” are used interchangeably.

The term “gene product” denotes molecules that are formed from the substrate of said genes by biochemical, chemical or physical reactions, such as DNA synthesis, transcription, splicing, translation, fragmentation or methylation. Preferred gene products of the invention are RNA, particularly mRNA and cRNA, cDNA and proteins.

As used herein, a “compound with estrogenic activity” is an agent that exerts at least some of the biological effects of estrogen, which refers to any factor, agent, compound whether endogenous or exogenous in origin, which is capable of binding and interacting with estrogen receptors and thereby eliciting certain biological effects of estrogen. The skilled artisan would know that, for instance, one of the biological effects of estrogen is to promote the development of the female reproductive system. Other biological effects of estrogen are well documented and discussed. For instance, estrogen is thought to affect tissues such as brain, liver, muscle, bone cells and stomach, which express the estrogen receptor gene. In the meaning of the invention, a “compound with anti-estrogenic activity” comprises such compounds that are able to reverse the estrogenic effects as described above.

“Estrogen” is a general term for hormones that are steroidal chemical substances secreted from ovarian follicles, placenta, and such and that induce the development of female reproductive organs such as follicles and mammary glands or other organs.

In the first step (a), a cellular system is provided. The cellular system is defined to be any subject provided that the subject comprises cells. Hence, the cellular system can be selected from the group of single cells, cell cultures, tissues, organs and mammals. The mammal is preferably a laboratory animal and/or a non-human organism. The scope of the cellular system also comprises parts of such biological entities, i.e. samples of tissues, organs and mammals. It shall be understood that each cellular system in the aforementioned order represents a sample of the respective following system.

Particularly, the cellular sample is taken in-vivo or in-situ from a mammal to be tested. The withdrawal of the cellular sample follows good medical practice. Biological samples may be taken from any kind of biological species, but the sample is especially taken from a human, rat or a mouse, more preferably a human. Such mammal should produce little or no estrogen if screening compounds with estrogenic activity. For instance, an aromatase knockout animal cannot produce estrogen. Because the major source of circulating estrogen is the ovary, ovariectomy dramatically decreases circulating estrogen levels. Thus, in one embodiment, ovariectomized animals are used. Contrary to that, an estrogen-stimulated cellular system is provided if screening compounds with anti-estrogenic activity.

In the present invention, the cellular system may also comprise a biological fluid, wherein the sample of body fluid preferably consists of blood, serum, plasma, saliva or urine. It is also preferred to gather a tissue sample by biopsy, especially taken close to the location of ailment. The biological samples can be originated from any tissue, including the uterus, pituitary gland, liver, brain, colon, breast, adipose tissue, etc. In preferred embodiments, the biological samples are from the kidney, pituitary gland and the uterus. The sample may be purified to remove disturbing substances, such as inhibitors for the formation of hydrogen bonds.

The cell sample refers to any type of primary cells or genetically engineered cells, either in the isolated status, in culture or as cell line, provided that they are capable of expressing at least one gene of Table 1. It shall be understood that variants, mutants, parts or homologous gene sequences having the same function, are included in the scope of definition as well as protection. The degree of alteration between the original sequence and its derivatives is inevitably limited by the requirement of altered gene expression by EACs. Preferably, the homology amounts to at least 85%. Possible alterations comprise deletion, insertion, substitution, modification and addition of at least one nucleotide, or the fusion with another nucleic acid. The engineered cells are capable of expressing these genes by transfection with appropriate vectors harboring them or parts thereof. Preferably, the recombinant cells are of eukaryotic origin.

In a more preferred embodiment of the present invention, the human Ishikawa cell line is provided in step (a) of the screening method. Ishikawa cells are human endometrial cancers cells of uterus origin.

The cell sample is stored, such as frozen, cultivated for a certain period or immediately subjected to step (b). Before incubating it with compounds to be screened, the cell sample is divided into multiple portions. At least two portions are provided; one is used for screening while the other one serves as control. Preferably, the number of portions for screening exceeds the number of control portions. Usually, numerous portions are subjected to a high-throughput screening.

The compounds are composed of biological and/or chemical structures capable to interact with a target molecule. Herein, any component of estrogen signaling shall be considered as “target molecule”, which is not limited to the estrogen receptor target, but may also comprise the selected genes themselves, or a regulator protein or a gene product thereof, or a component of a signal transduction pathway comprising said gene or gene products thereof. Consequently, the specific interaction of compounds may involve either the mere targeting or the induction of alterations in cell function, or it may even include both effects simultaneously.

The compounds to be screened in the inventive method are not restricted anyway. In particular, the compounds are selected from the group of nucleic acids, peptides, carbohydrates, polymers, small molecules having a molecular weight between 50 and 1.000 Da and proteins. These compounds are often available in libraries. It is preferred to incubate a single compound within a distinct portion of the cell sample. However, it is also possible to investigate the cooperative effect of compounds by incubating at least two compounds within one portion. A further portion of cells is simultaneously incubated in the absence of the compounds.

The term “incubation” denotes the contacting of the compounds with the cells for a distinct period, which depends on the kind of compounds and/or target. The incubation process also depends on various other parameters, e.g. the cell type and the sensitivity of detection, which optimization follows routine procedures known to those skilled in the art. The incubation procedure can be realized without a chemical conversion or may involve a biochemical reaction. Adding chemical solutions and/or applying physical procedures, e.g. impact of heat, can improve the accessibility of the target structures in the sample. Specific incubation products are formed as result of the incubation.

In step (c), the identification of effective compounds in the meaning of the invention is indirectly performed by determining the expression pattern of at least a single gene of Table 1, which the system is capable of expressing. The determination is performed at a specified moment and correlated to the signal strength at the beginning of the experiment and the positive/negative control. Either the control system is not incubated with the compounds (negative control) or the control system is incubated with a standard compound having no estrogenic/anti-estrogenic activity (negative control) or a standard compound having estrogenic/anti-estrogenic activity (positive control) as set forth at the example of microarray below The activity is revealed by a change in expression. Preferably, the genes expressed or repressed in cells with EAC exposure are compared to the genes expressed or repressed in cells that were not exposed to EAC. Pairwise comparisons are made between each of the treatments. A pairwise comparison is the expression data for a given gene under a given treatment condition compared to the expression data for this gene under a second treatment condition. The comparison is performed using suitable statistical technique with the assistance of known and commercially available programs.

Suitable tests for monitoring gene expression, determination and variant analysis of nucleotide sequences are known to those skilled in the art or can be easily designed as a matter of routine. The assay according to the invention may be any assay suitable to detect and/or quantify gene expression. Many different types of assays are known, examples of which are set forth below, including analyses by nucleotide arrays and nucleotide filters. The hybridization conditions (temperature, time, and concentrations) are adjusted according to procedures also well known in the art. It is preferred to apply chip hybridization and/or PCR for the determination of gene expression. In another preferred embodiment, the assay of the invention involves the use of a high density oligonucleotide array. In still another preferred embodiment, the analysis is performed by multiplex qPCR, more preferably low density TaqMan arrays or branched DNA assays. Other solid supports and microarrays are known and commercially available to the skilled artisan.

Consequently, this invention relates to a method for predicting the cellular effect of a compound having estrogen-like activity by preparing a nucleic acid sample from a cell to be evaluated, contacting the nucleic acid sample with an microarray, detecting a nucleic acid hybridizing with the microarray, and comparing a result detected in step (c) with a result detected using a nucleic acid sample prepared from a control cell.

In a preferred embodiment of the present invention, the gene products RNA, cRNA, cDNA and/or protein are detected, more preferably mRNA, cRNA and/or cDNA. For instance, the total RNA from such cells is prepared by methods known to the skilled artisan such as by Trizol (Invitrogen) followed by subsequent re-purification, e.g. via Rneasy columns (Qiagen).

The total RNA is used to generate a labeled target according to methods and using detectable labels well-know in the art. For instance, the RNA may be labeled with biotin to form a cRNA target for use in an assay. Next, with the extracted mRNA as a template, cDNAs are produced using a reverse transcriptase (for example, SuperScript Reverse Transcriptase; GibcoBRL) and labeled dNTP (for example, Cy3-dUTP and Cy5-dUTP; Amersham Pharmacia Biotech), and a cDNA sample that reflects the amount of genes expressed within the cells to be evaluated is prepared. This causes labeled cDNA to be included in the cDNA sample. Here, either fluorescent label or radiolabel may be used as a label. The cDNA sample prepared in this manner is applied to the below-mentioned microarray in its single stranded denatured form, and cDNAs included in the cDNA sample are hybridized with the genes immobilized on the basal plate.

“In-situ hybridization” is a methodology for determining the presence of or the copy number of a gene in a sample, for example, fluorescence in-situ hybridization (FISH). Generally, in-situ hybridization comprises the following major steps: (1) fixation of tissue or biological structure to be analyzed; (2) pre-hybridization treatment of the biological structure to increase accessibility of target nucleic acid, and to reduce non-specific binding; (3) hybridization of the mixture of nucleic acids to the nucleic acid in the biological structure or tissue; (4) post-hybridization washes to remove nucleic acid fragments not bound in the hybridization; and (5) detection of the hybridized nucleic acid fragments. The probes used in such applications are typically labeled, for example, with radioisotopes or fluorescent reporters. Preferred probes are sufficiently long, for example, from about 50, 100 or 200 nucleotides (nt) to about 1000 or more nucleotides, to enable specific hybridization with the target nucleic acid(s) under stringent conditions. Here, hybridization with cDNA can be accomplished, for example, by incubating at 65° C. for 10 to 20 hours.

As used herein, the term “microarray” refers to nucleotide arrays that can be used to detect biomolecules, for instance to measure gene expression. “Array”, “slide” and “(DNA) chip” are used interchangeably in this disclosure. A microarray usually comprises a basal plate, e.g. made of slide glass, silicone, or the like, and DNA fragments immobilized as an array on this basal plate. With this microarray, DNAs contained in a sample can be detected by hybridizing them with the DNA fragments immobilized on the basal plate. Since the DNA within the sample is radiolabeled or fluorescently labeled, detection with radio imaging scanner, fluorescence imaging scanner, or the like is possible. Various kinds of arrays are made in research and manufacturing facilities worldwide, some of which are available commercially. There are, for example, two main kinds of nucleotide arrays that differ in the manner in which the nucleic acid materials are placed onto the array substrate: spotted arrays and in-situ synthesized arrays. One of the most widely used oligonucleotide arrays is GeneChip made by Affymetrix, Inc. The oligonucleotide probes have a length of 10 to 50 nucleotides (nt), preferably 15 to 30 nt, more preferably 20 to 25 nt. They are synthesized in-silico on the array substrate. These arrays tend to achieve high densities, e.g. more than 40,000 genes per cm². The spotted arrays, on the other hand, tend to have lower densities, but the probes, typically partial cDNA molecules, usually are much longer than 25 nucleotides. A representative type of spotted cDNA array is LifeArray made by Incyte Genomics. Pre-synthesized and amplified cDNA sequences are attached to the substrate of these kinds of arrays.

In one embodiment, the array is a matrix, in which each position represents a discrete binding site for a product encoded by a gene, e.g. a protein or RNA, and in which binding sites are present for products of most or almost all of the genes according to Table 1 and optionally Table 4. In one embodiment, the “binding site” (hereinafter “site”) is a nucleic acid or nucleic acid analogue to which a particular cognate cDNA can specifically hybridize. The nucleic acid or analogue of the binding site can be, e.g. a synthetic oligomer, a full-length cDNA, a less-than full length cDNA or a gene fragment. Preferably, the microarray has binding sites for genes relevant to the action of the gene expression modulating agent of interest or in a biological pathway of interest. It is preferably that more than one DNA fragment, which is capable of hybridizing under stringent conditions to a gene or parts thereof as selected from the group of genes according to Table 1 and optionally Table 4, is immobilized on the basal plate. The DNA fragment to be immobilized on the basal plate may contain the whole or a part of the genes. The term “parts of a gene” used herein means a portion of the gene and a nucleotide sequence equivalent to at least 10 nt, preferably at least 25 nt, more preferably 50 nt, most preferably 300 nt, highly preferably 500 nt.

It is additionally preferable that genes constitutively expressing regardless of the presence or absence of chemical substances having estrogen-like activity (hereinafter referred to as negative control genes and the like) are immobilized on the basal plates of the microarray. The expression level of the genes according to the invention can be corrected by immobilizing negative control genes on the basal plate and correcting the expression level of the negative control genes to a constant value. Thus, the changes in the expression level of genes according to Table 1 and optionally Table 4 can be detected with certainty. Accuracy can be further enhanced by choosing several negative control genes and/or such that have different expression levels.

The nucleic acid or analogue are attached to a solid support or basal plate, which terms are used interchangeably herein, and which may be made from glass, plastic (e.g. polypropylene or nylon), polyacrylamide, nitrocellulose or other materials. When the DNA fragments and negative control genes are immobilized on the basal plate, a conventionally known technique can be used. For example, the surface of the basal plate can be treated with polycations such as polylysines to electrostatically bind the DNA fragments through their charges on the surface of the basal plate. Furthermore, techniques to covalently bind the 5′-end of the DNA fragments to the basal plate may be used. Alternatively, a basal plate having linkers on its surface can be produced, and functional groups that can form covalent bonds with the linkers are introduced at the end of the DNA fragments. The DNA fragments are immobilized by forming a covalent bond between the linker and the functional group. A preferred method for attaching the nucleic acids to a surface is by printing on glass plates.

Finally, cDNAs that hybridized with the DNA fragments on the microarray are detected. In cases where the hybridized cDNAs are fluorescently labeled, the fluorescence is detected with, for example, a fluorescence laser microscope and a CCD camera, and the fluorescence intensity is analyzed with a computer. Similarly, in cases where the hybridized cDNAs are radiolabeled, detection can be carried out using an RI image scanner and such, and the intensity of the radiation can be analyzed with a computer.

In another embodiment of the screening method, the detection of estrogenic or anti-estrogenic activity can be additionally refined in step (c). For this purpose, the gene expression is determined by detecting at least one gene product encoded by the gene(s) of Table 1 and correlating an amount of signal or change in signal with the gene expression in the system. The cellular system of the invention is incubated with various concentrations of an identified endocrine active compound. The amount of emitted signal or change in signal observed in the presence of the EAC is indicative of the change in gene expression experienced by the compound. The change can be then related to the concentration of the EAC in the sample, i.e. the calibration curve enables the meter-reading of a matching concentration. Preferably, the calibration curve is based on the Lambert-Beer equation if using UV/VIS coloring or luminescence. Estrogenicity of compounds is diagnosed by comparing the concentration of the gene product in the sample with known gene product concentration levels of either non-estrogenic cells and/or estrogenic cells. It shall be understood that the known concentrations are statistically proven, therefore representing a certain level or range, respectively. The direction and strength of gene expression have also been figured out by the differential expression analysis of the target genes of the invention such that either a distinct up-regulation or down-regulation with a certain factor has been recognized as set forth below, which forms the basis of biomarker selection. Any measured concentration, which differs from the gene product concentration level of EAC-unstimulated cells, indicates an abnormality of the tested cell sample, whereas a compound cannot be classified as EAC at a gene product concentration which is comparable to the concentration level of EAC-unstimulated cells. It is preferred to measure concentration, which are higher than the gene product concentration level of unstimulated cells, for detecting estrogenicity. Using this method, the inventors demonstrated sensitivity to submicromolar or even nanomolar concentrations. The calibration plot reveals that the method can be applied in a dynamic range that spans over a couple of magnitude.

According to a preferred embodiment of the invention, the “Polymerase Chain Reaction” or “PCR” is an amplification-based assay used to measure the copy number of the gene. In such assays, the corresponding nucleic acid sequences act as a template in an amplification reaction. In a quantitative amplification, the amount of amplification product will be proportional to the amount of template in the original sample. Comparison to appropriate controls provides a measure of the copy number of the gene, corresponding to the specific probe used, according to the principle discussed above.

Detailed protocols for real-time quantitative PCR are provided, for example, for RNA. The “level of mRNA” in a biological sample refers to the amount of mRNA transcribed from a given gene that is present in a cell or a biological sample. One aspect of the biological state of a biological sample (e.g. a cell or cell culture) usefully measured in the present invention is its transcriptional state. The transcriptional state of a biological sample includes the identities and abundances of the constituent RNA species, especially mRNAs, in the cell under a given set of conditions. Preferably, a substantial fraction of all constituent RNA species in the biological sample are measured, but at least a sufficient fraction is measured to characterize the action of a compound of interest.

The primers are designed based on the nucleotide sequence information of the region flanking the site to be amplified. The primers may be designed so as to amplify a region of 100 to 200 base pairs in length. The nucleic acid amplification method includes, but is not particularly limited to, a PCR, preferably a real-time PCR. The level of mRNA may also be quantified by other methods described herein.

After performing an amplification reaction of a nucleic acid using the biological sample to be analyzed and primers as described above, it is checked whether the nucleic acid is amplified or not. In order to facilitate the detection of an amplified nucleic acid, a primer may be labeled in advance. Examples of applicable fluorescent labels include FAM™, TET™, HEX™, TAMRA™ and ROX™ manufactured by Applied Biosystems. In these cases, either the 5′-end or the 3′-end of a primer may be labeled, preferably the 5′-end. Alternatively, the nucleic acid may be labeled during PCR by using labeled nucleotides, or even after PCR is completed. Light emission is measured by a general-purpose luminescence determination device.

Methods of “real-time quantitative PCR” using TaqMan probes are also well-known in the art. Hence, a TaqMan-based assay can be applied to quantify polynucleotides. TaqMan based assays use a fluorogenic oligonucleotide probe that contains a 5′-fluorescent dye and a 3′-quenching agent. The probe hybridizes to a PCR product, but cannot itself be extended due to a blocking agent at the 3′-end. When the PCR product is amplified in subsequent cycles, the 5′-nuclease activity of the polymerase, for example, AmpliTaq, results in the cleavage of the TaqMan probe. This cleavage separates the 5′-fluorescent dye and the 3′-quenching agent, thereby resulting in an increase in fluorescence as a function of amplification.

Other suitable amplification methods include, but are not limited to, ligase chain reaction (LCR), transcription amplification, self-sustained sequence replication, dot PCR and linker adapter PCR.

When mass spectroscopy is used, primers may be designed so as to allow the amplification of nucleic acid fragments having a length that varies with the expression pattern.

The presence or absence of an amplified nucleic acid fragment can also be checked by subjecting a reaction solution to electrophoresis, such as for single-strand conformation polymorphism (SSCP) analysis, which may be performed by capillary electrophoresis. However, other electrophoresis methods, for instance gel electrophoresis, are also applicable and well known to those skilled in the art.

Therefore, the present invention relates to the assessment or measurement of modulations of gene expression by the assays as set forth above. Such modulation refers to the induction or inhibition of expression of a gene. Typically, modulation of gene expression may be caused by endogenous or exogenous factors or agents. The effect of a given compound can be measured by any means known to those skilled in the art. For example, expression levels may be measured by PCR, Northern blotting, Primer Extension, Differential Display techniques, etc. The induction of expression (i.e. up-regulation) refers to any observable or measurable increase in the levels of expression of a particular gene, either qualitatively or quantitatively. Contrary to that, the inhibition of expression (i.e. down-regulation) refers to any observable or measurable decrease in the levels of expression of a particular gene, either qualitatively or quantitatively. The measurement of levels of expression may be carried out using any techniques that are capable of measuring RNA transcripts in a biological sample. Examples of these techniques include, as discussed above, PCR, TaqMan, Primer Extension, Differential display and nucleotide arrays, among other things. It is another embodiment of the present invention that in the case of modulation the gene product concentration either exceeds or under-run, respectively, at least twice the gene product concentration in the control system, preferably at least 10 times, more preferably at least 25 times, most preferably at least 40 times

In a preferred embodiment of the invention, the estrogenic activity of a compound is positively detected in step (c) if the expression involves an up-regulation of genes selected from the group of genes according to Table 2 and/or a down-regulation of genes selected from the group of genes according to Table 3. The term “positive detection” denotes to the fact that the respective activity is actually proven as inherent feature of a certain compound. Several novel genes, not characterized in conjunction with estrogen receptor activity, but involved in positive regulation of cell growth and development, were identified as being markedly up-regulated by estrogenic compounds (Table 2) but inhibited by ICI. PIM1, a proto-oncogene having intrinsic serine-threonine kinase activity, can enhance cell cycle progression by altering the activity of several cell cycle regulators including p21 (Waf), Cdc25A and C-TAK1. G0/G1 switch 2 (G0S2) was found to be involved in adipocyte differentiation and drives cell cycle progression in blood mononuclear cells. Aminopeptidase N (ANPEP) and the structural molecule Laminin beta 3 (LAMB3) play an important role in cell migration during tumor invasion and tissue remodeling. Amino acid transporters, for example L-type amino acid transporter LAT1 (SCL7A5) and its subunit 42F heavy chain 42F hc (SCL3A2), function in supplying essential amino acids to cells which are required for protein synthesis and as energy sources. High expression of both of these genes was found in tumor cells and promoted tumor growth progression. Furthermore, SLC3A2 can specifically associate with beta 1 integrins on the surface of human tumor cells and therefore contributes to malignant transformation, by allowing anchorage and serum-independent-growth. Among marker genes which were down-regulated in response to estrogenic chemicals (Table 3), most were found to be related to cell growth inhibition (spermidine/spermine N1-acetyltransferase, SAT, and retinol binding protein 1 cellular, RBP1) and apoptosis induction (proline dehydrogenase oxidase 1, PRODH and tumor necrosis factor receptor superfamily member 25, TNFRSF25).

Contrary to that, the anti-estrogenic activity of a compound is positively detected in step (c) if the expression involves a down-regulation of genes of Table 2 and/or an up-regulation of genes of Table 3.

Although each biomarker gene of the invention exhibits a sensitivity that allows the use of a single marker gene in the scope of the screening method, it is preferred to apply more than one marker gene for detecting estrogenicity. In an embodiment of the invention, the cellular system provided in step (a) is therefore capable of expressing at least two genes of Table 1, preferably at least 10 genes, more preferably at least 25 genes, most preferably at least 40 genes, highly preferably the entire panel of 72 genes. Accordingly, the expression of at least two genes of Table 1 is compared with the gene expression in the control system in step (c), preferably at least 10 genes, more preferably at least 25 genes, most preferably at least 40 genes, highly preferably the entire panel of 72 genes. The inventors have illustrated that analyzing multiple estrogen-responsive genes increases screening stability and reduces error rates by covering a broader spectrum of estrogenic responses than single-gene reporter assays. The prior teaching concerning multiple genes is valid and applicable without restrictions to the Tables 2 and 3, which represents subsets of Table 1, provided that the respective preferred plurality of genes is re-calculated by the rule of three.

In addition to the expression of genes, which are selected from the group according to Table 1, the cellular system or the sample thereof is preferably capable of expressing at least a single gene of Table 4 in step (a) of the inventive screening method. Furthermore, in step (c) the expression of the single gene of Table 4 is compared with the gene expression in the control system. Several estrogen/estrogen receptor-regulated genes were identified, such as alkaline phosphatase placental-like 2 (ALPPL2), progesterone receptor (PGR), seven in absentia homolog 2 (Drosophila) (SIAH2), transforming growth factor alpha (TGFA) and genes modulating estrogen receptor activity, such as nuclear receptor interacting protein 1 (NRIP1). Moreover, a pathway of note, WNT/β-catenin signaling, was up-regulated by most estrogenic compound treatments but not after ICI treatment. WNT/β-catenin signaling is involved in a variety of developmental processes including regulation of cell growth and differentiation. WNT2B encodes a member of the wingless-type MMTV integration site family of highly conserved, secreted signaling molecules. Up-regulation of WNT2B by estrogen might play an important role in human breast cancer. The HMG box transcription factor, SOX17, can interact with β-catenin and potentiates the transcriptional activation of target genes similar to Tcf/Lef. Four and a half LIM domains 2 (FHL2) protein exerts co-activator properties enhancing β-catenin/TCF-mediated transcription and was found to be over-expressed in ovarian cancer.

In a preferred embodiment of the invention, the estrogenic activity of a compound is positively detected in step (c) if the expression involves an up-regulation of genes selected from the group of genes according to Table 5 and/or a down-regulation of genes selected from the group of genes according to Table 6. Contrary to that, the anti-estrogenic activity of a compound is positively detected in step (c) if the expression involves a down-regulation of genes of Table 5 and/or an up-regulation of genes of Table 6.

In another preferred embodiment of the invention, multiple genes of Table 4 are applied in both steps (a) and (c), more preferably at least 2 genes, most preferably at least 10 genes, highly preferably the entire panel of 15 genes. Once more, the prior teaching concerning multiple genes is valid and applicable without restrictions to the Tables 5 and 6, which represents subsets of Table 4, provided that the respective preferred plurality of genes is re-calculated by the rule of three.

In a more preferred embodiment of the invention, the expression of the selected estrogenic marker genes ALPP2, CEBPD, FOXD1, GOS2, NRIP1, PGR and PIM1 is compared with the gene expression in the control system. In a most preferred embodiment of the invention, the expression of all genes of Table 1 and Table 4 is compared with the gene expression in the control system. The identified 87 genes showed similar expression patterns in response to all EAC treatments in Ishikawa plus, whereas ICI lowered the magnitude or reversed the expression of these genes, indicating ER dependent regulation. Apart from estrogenic gene regulation Bisphenol A, o,p′-DDT, Resveratrol, Zearalenone and Genistein displayed similarities to ICI in their expression patterns.

In another preferred embodiment of the invention, the cellular system provided in step (a) is capable of expressing at least one gene that is selected from the group of the particular preferred genes according to Table 7. Accordingly, the expression of the at least one gene of Table 7 is compared with the gene expression in the control system in step (c). It is also preferred to apply a plurality of genes according to Table 7 in both steps (a) and (c), more preferably at least 2 genes, most preferably at least 10 genes, highly preferably the entire panel of 49 genes. In a more preferred embodiment of the invention, the estrogenic activity of a compound is positively detected in step (c) if the expression involves an up-regulation of genes selected from the group of genes according to Table 8 and/or a down-regulation of genes selected from the group of genes according to Table 9. Contrary to that, the anti-estrogenic activity of a compound is positively detected in step (c) if the expression involves a down-regulation of genes of Table 8 and/or an up-regulation of genes of Table 9. The prior teaching concerning multiple genes is valid and applicable without restrictions to the Tables 8 and 9, which represents subsets of Table 7, provided that the respective preferred plurality of genes is re-calculated by the rule of three.

In addition to the expression of genes of Table 7, the cellular system of step (a) is preferably capable of expressing at least a single gene, which is selected from the group of genes according of Table 10, whose expression is compared with the gene expression in the control system in step (c). It is also preferred to apply a plurality of genes according to Table 10 in both steps (a) and (c), more preferably at least 2 genes, most preferably at least 10 genes, highly preferably the entire panel of 34 genes. In a preferred embodiment of the invention, the estrogenic activity of a compound is positively detected in step (c) if the expression involves an up-regulation of genes selected from the group of genes according to Table 11 and/or a down-regulation of genes selected from the group of genes according to Table 12. Contrary to that, the anti-estrogenic activity of a compound is positively detected in step (c) if the expression involves a down-regulation of genes of Table 11 and/or an up-regulation of genes of Table 12. The prior teaching concerning multiple genes is valid and applicable without restrictions to the Tables 11 and 12, which represents subsets of Table 10, provided that the respective preferred plurality of genes is re-calculated by the rule of three.

If in step (a) the cellular system or the sample thereof is capable of expressing multiple genes of Table 1 and/or additionally capable of expressing multiple genes of Table 4, and furthermore in step (c) an expression pattern of multiple genes of Table 1 and/or Table 4 is compared with the expression pattern in the control system, the estrogenicity can be characterized compound-specifically. Particularly, the expression pattern is determined by a correlation of the multiple genes and/or a magnitude of altered regulation. The screening method of this invention not only evaluates the effect of chemical substances having estrogen-like or anti-estrogenic activity on cells to be evaluated, but can also indicate the details of this effect. By individually evaluating the expression level of categorized genes, it is possible to distinguish how chemical substances having estrogen-like or anti-estrogenic activity that affect the cells to be evaluated.

The invention also teaches an embodiment of the screening method, wherein in step (a) a mammal, preferably a laboratory mammal, is provided, in step (b) the compound to be screened is administered to the mammal, and in step (c) a therapeutic effect is detected via a level of estrogenic or anti-estrogenic activity in a biological sample withdrawn from the mammal in comparison with a mammal showing non-endocrine disrupting and/or endocrine disrupting effects. With the therapeutic effect, the qualitative level is incorporated into step (c). A “therapeutically relevant effect” relieves to some extent one or more symptoms of a disease or returns to normality, either partially or completely, one or more physiological or biochemical parameters associated with or causative of the disease or pathological conditions. In addition, the expression “therapeutically effective amount” denotes an amount which, compared with a corresponding subject who has not received this amount, has the following consequence: improved treatment, healing, prevention or elimination of a disease, syndrome, condition, complaint, disorder or side-effects or also the reduction in the advance of a disease, complaint or disorder. The expression “therapeutically effective amount” also encompasses the amounts which are effective for increasing normal physiological function. Testing of several compounds makes the selection of that compound possible that is best suited for the treatment of the mammal subject. The in-vivo dose rate of the chosen compound is advantageously pre-adjusted to the estrogenicity of the specific cells with regard to their in-vitro data. Therefore, the therapeutic efficacy is remarkably enhanced.

A compound identified by the screening method is another object of the invention. The prior teaching of the present specification concerning the screening method is valid and applicable without restrictions to the compound itself if expedient.

The identification of compounds that induce or repress the expression of a gene associated with a given disorder or condition can lead to the development of pharmaceuticals that can be administered to a patient at therapeutically effective doses to prevent, treat or control such disorder or condition. Hence, the invention furthermore relates to a medicament comprising at least one compound according to the invention, and optionally excipients and/or adjuvants. In the meaning of the invention, an “adjuvant” denotes every substance that enables, intensifies or modifies a specific response against the active ingredient of the invention if administered simultaneously, contemporarily or sequentially. Known adjuvants for injection solutions are, for example, aluminum compositions, such as aluminum hydroxide or aluminum phosphate, saponins, such as QS21, muramyldipeptide or muramyltripeptide, proteins, such as gamma-interferon or TNF, M59, squalen or polyols. Consequently, the invention also relates to a pharmaceutical composition comprising as active ingredient an effective amount of at least one compound as screened according to the invention and/or physiologically acceptable salts thereof together with pharmaceutically tolerable adjuvants.

A “medicament”, “pharmaceutical composition” or “pharmaceutical formulation” in the meaning of the invention is any agent in the field of medicine, which comprises one or more EAC of the invention or preparations thereof and can be used in prophylaxis, therapy, follow-up or aftercare of patients who suffer from diseases, which are associated with estrogen receptor signaling, in such a way that a pathogenic modification of their overall condition or of the condition of particular regions of the organism could establish at least temporarily.

Furthermore, the active ingredient may be administered alone or in combination with other treatments. A synergistic effect may be achieved by using more than one compound in the pharmaceutical composition, i.e. the EAC of the invention is combined with at least another agent as active ingredient. The active ingredients can be used either simultaneously or sequentially.

Pharmaceutical formulations can be adapted for administration via any desired suitable method, for example by oral (including buccal or sublingual), rectal, nasal, topical (including buccal, sublingual or transdermal), vaginal or parenteral (including subcutaneous, intramuscular, intravenous or intradermal) methods. Such formulations can be prepared using all processes known in the pharmaceutical art by, for example, combining the active ingredient with the excipient(s) or adjuvant(s).

The pharmaceutical composition of the invention is produced in a known way using common solid or liquid carriers, diluents and/or additives and usual adjuvants for pharmaceutical engineering and with an appropriate dosage. The amount of excipient material that is combined with the active ingredient to produce a single dosage form varies depending upon the host treated and the particular mode of administration. Suitable excipients include organic or inorganic substances that are suitable for the different routes of administration, such as enteral (e.g. oral), parenteral or topical application, and which do not react with compounds of the invention or salts thereof. Examples of suitable excipients are water, vegetable oils, benzyl alcohols, alkylene glycols, polyethylene glycols, glycerol triacetate, gelatin, carbohydrates, such as lactose or starch, magnesium stearate, talc, and petroleum jelly.

Pharmaceutical formulations adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

Thus, for example, in the case of oral administration in the form of a tablet or capsule, the active-ingredient component can be combined with an oral, non-toxic and pharmaceutically acceptable inert excipient, such as, for example, ethanol, glycerol, water and the like. Powders are prepared by comminuting the compound to a suitable fine size and mixing it with a pharmaceutical excipient comminuted in a similar manner, such as, for example, an edible carbohydrate, such as, for example, starch or mannitol. A flavor, preservative, dispersant and dye may likewise be present.

Capsules are produced by preparing a powder mixture as described above and filling shaped gelatin shells therewith. Glidants and lubricants, e.g. highly disperse silicic acid, talc, magnesium stearate, calcium stearate or polyethylene glycol in solid form, can be added to the powder mixture before the filling operation. A disintegrant or solubiliser, such as, for example, agar-agar, calcium carbonate or sodium carbonate, may likewise be added in order to improve the availability of the medicament after the capsule has been taken.

In addition, if desired or necessary, suitable binders, lubricants and disintegrants as well as dyes can likewise be incorporated into the mixture. Suitable binders include starch, gelatin, natural sugars, such as, for example, glucose or beta-lactose, sweeteners made from maize, natural and synthetic rubber, such as, for example, acacia, tragacanth or sodium alginate, carboxymethylcellulose, polyethylene glycol, waxes, and the like. The lubricants used in these dosage forms include sodium oleate, sodium stearate, magnesium stearate, sodium benzoate, sodium acetate, sodium chloride and the like. The disintegrants include, without being restricted thereto, starch, methylcellulose, agar, bentonite, xanthan gum and the like. The tablets are formulated by, for example, preparing a powder mixture, granulating or dry-pressing the mixture, adding a lubricant and a disintegrant and pressing the entire mixture to give tablets. A powder mixture is prepared by mixing the compound comminuted in a suitable manner with a diluent or a base, as described above, and optionally with a binder, such as, for example, carboxymethylcellulose, an alginate, gelatin or polyvinylpyrrolidone, a dissolution retardant, such as, for example, paraffin, an absorption accelerator, such as, for example, a quaternary salt, and/or an absorbent, such as, for example, bentonite, kaolin or dicalcium phosphate. The powder mixture can be granulated by wetting it with a binder, such as, for example, syrup, starch paste, acadia mucilage or solutions of cellulose or polymer materials and pressing it through a sieve. As an alternative to granulation, the powder mixture can be run through a tableting machine, giving lumps of non-uniform shape, which are broken up to form granules. The granules can be lubricated by addition of stearic acid, a stearate salt, talc or mineral oil in order to prevent sticking to the tablet casting moulds. The lubricated mixture is then pressed to give tablets. The compounds according to the invention can also be combined with a free-flowing inert excipient and then pressed directly to give tablets without carrying out the granulation or dry-pressing steps. A transparent or opaque protective layer consisting of a shellac sealing layer, a layer of sugar or polymer material and a gloss layer of wax may be present. Dyes can be added to these coatings in order to be able to differentiate between different dosage units.

Oral liquids, such as, for example, solution, syrups and elixirs, can be prepared in the form of dosage units so that a given quantity comprises a pre-specified amount of the compound. Syrups can be prepared by dissolving the compound in an aqueous solution with a suitable flavor, while elixirs are prepared using a non-toxic alcoholic vehicle. Suspensions can be formulated by dispersion of the compound in a non-toxic vehicle. Solubilisers and emulsifiers, such as, for example, ethoxylated isostearyl alcohols and polyoxyethylene sorbitol ethers, preservatives, flavor additives, such as, for example, peppermint oil or natural sweeteners or saccharin, or other artificial sweeteners and the like, can likewise be added.

The dosage unit formulations for oral administration can, if desired, be encapsulated in microcapsules. The formulation can also be prepared in such a way that the release is extended or retarded, such as, for example, by coating or embedding of particulate material in polymers, wax and the like.

The compounds according to the invention and salts, solvates and physiologically functional derivatives thereof can be administered in the form of liposome delivery systems, such as, for example, small unilamellar vesicles, large unilamellar vesicles and multilamellar vesicles. Liposomes can be formed from various phospholipids, such as, for example, cholesterol, stearylamine or phosphatidylcholines.

The active ingredient according to the invention can also be fused or complexed with another molecule that promotes the directed transport to the destination, the incorporation and/or distribution within the target cells.

The compounds according to the invention and the salts, solvates and physiologically functional derivatives thereof can also be delivered using monoclonal antibodies as individual carriers to which the compound molecules are coupled. The compounds can also be coupled to soluble polymers as targeted medicament carriers. Such polymers may encompass polyvinylpyrrolidone, pyran copolymer, polyhydroxypropylmethacrylamido-phenol, polyhydroxyethylaspartamidophenol or polyethylene oxide polylysine, substituted by palmitoyl radicals. The compounds may furthermore be coupled to a class of biodegradable polymers which are suitable for achieving controlled release of a medicament, for example polylactic acid, poly-epsilon-caprolactone, polyhydroxybutyric acid, polyorthoesters, polyacetals, polydihydroxypyrans, polycyanoacrylates and crosslinked or amphipathic block copolymers of hydrogels.

Pharmaceutical formulations adapted for transdermal administration can be administered as independent plasters for extended, close contact with the epidermis of the recipient. Thus, for example, the active ingredient can be delivered from the plaster by iontophoresis, as described in general terms in Pharmaceutical Research, 3 (6), 318 (1986).

Pharmaceutical compounds adapted for topical administration can be formulated as ointments, creams, suspensions, lotions, powders, solutions, pastes, gels, sprays, aerosols or oils.

For the treatment of the eye or other external tissue, for example mouth and skin, the formulations are preferably applied as topical ointment or cream. In the case of formulation to give an ointment, the active ingredient can be employed either with a paraffinic or a water-miscible cream base. Alternatively, the active ingredient can be formulated to give a cream with an oil-in-water cream base or a water-in-oil base.

Pharmaceutical formulations adapted for topical application to the eye include eye drops, in which the active ingredient is dissolved or suspended in a suitable carrier, in particular an aqueous solvent.

Pharmaceutical formulations adapted for topical application in the mouth encompass lozenges, pastilles and mouthwashes.

Pharmaceutical formulations adapted for rectal administration can be administered in the form of suppositories or enemas.

Pharmaceutical formulations adapted for nasal administration in which the carrier substance is a solid comprise a coarse powder having a particle size, for example, in the range 20-500 microns, which is administered in the manner in which snuff is taken, i.e. by rapid inhalation via the nasal passages from a container containing the powder held close to the nose. Suitable formulations for administration as nasal spray or nose drops with a liquid as carrier substance encompass active-ingredient solutions in water or oil.

Pharmaceutical formulations adapted for administration by inhalation encompass finely particulate dusts or mists, which can be generated by various types of pressurized dispensers with aerosols, nebulisers or insufflators.

Pharmaceutical formulations adapted for vaginal administration can be administered as pessaries, tampons, creams, gels, pastes, foams or spray formulations.

Pharmaceutical formulations adapted for parenteral administration include aqueous and non-aqueous sterile injection solutions comprising antioxidants, buffers, bacteriostatics and solutes, by means of which the formulation is rendered isotonic with the blood of the recipient to be treated; and aqueous and non-aqueous sterile suspensions, which may comprise suspension media and thickeners. The formulations can be administered in single-dose or multi-dose containers, for example sealed ampoules and vials, and stored in freeze-dried (lyophilized) state, so that only the addition of the sterile carrier liquid, for example water for injection purposes, immediately before use is necessary. Injection solutions and suspensions prepared in accordance with the recipe can be prepared from sterile powders, granules and tablets.

It goes without saying that, in addition to the above particularly mentioned constituents, the formulations may also comprise other agents usual in the art with respect to the particular type of formulation; thus, for example, formulations which are suitable for oral administration may comprise flavors.

In a preferred embodiment of the present invention, the pharmaceutical composition is orally or parenterally administered, more preferably orally. In particular, the active ingredient is provided in a water-soluble form, such as a pharmaceutically acceptable salt, which is meant to include both acid and base addition salts. Furthermore, the compounds of the invention and salts thereof may be lyophilized and the resulting lyophilizates used, for example, to produce preparations for injection. The preparations indicated may be sterilized and/or may comprise auxiliaries, such as carrier proteins (e.g. serum albumin), lubricants, preservatives, stabilizers, fillers, chelating agents, antioxidants, solvents, bonding agents, suspending agents, wetting agents, emulsifiers, salts (for influencing the osmotic pressure), buffer substances, colorants, flavorings and one or more further active substances, for example one or more vitamins. Additives are well known in the art, and they are used in a variety of formulations.

Pharmaceutical formulations can be administered in the form of dosage units which comprise a predetermined amount of active ingredient per dosage unit. The concentration of the prophylactically or therapeutically active ingredient in the formulation may vary from about 0.1 to 100 wt %. Preferably, the compound of formula (I) or the pharmaceutically acceptable salts thereof are administered in doses of approximately 0.5 to 1000 mg, more preferably between 1 and 700 mg, most preferably 5 and 100 mg per dose unit. Generally, such a dose range is appropriate for total daily incorporation. In other terms, the daily dose is preferably between approximately 0.02 and 100 mg/kg of body weight. The specific dose for each patient depends, however, on a wide variety of factors (e.g. depending on the condition treated, the method of administration and the age, weight and condition of the patient). Preferred dosage unit formulations are those which comprise a daily dose or part-dose, as indicated above, or a corresponding fraction thereof of an active ingredient. Furthermore, pharmaceutical formulations of this type can be prepared using a process which is generally known in the pharmaceutical art.

The invention also relates to a method for monitoring physiological and/or pathological conditions, which are caused, mediated and/or propagated by estrogen receptor signaling, wherein an effective amount of at least one compound or a physiologically acceptable salt thereof is administered to a mammal in need of such treatment and an expression of at least one gene of Table 1 is determined in a biological sample withdrawn from the mammal. The compound is preferably obtained by the screening method of the invention as set forth above. Thus, the prior teaching of the present specification concerning the screening method is valid and applicable without restrictions to method of monitoring if expedient.

The identification of the plurality of genes described above provides a powerful tool for assessing the progression of a state, condition or treatment. Specifically, a plurality of genes can be identified in a patient prior to an event, such as menopause, surgery, the onset of a therapeutic regime, or the completion of a ‘therapeutic regime, to provide a base line result. This base-line can then be compared with the result obtained using identical methods either during or after such event. This information can be used for both diagnostic and prognostic purposes.

The inventive method of monitoring can be employed in human and veterinary medicine. Herein, the compounds can be administered before or following an onset of disease once or several times acting as therapy. The terms “effective amount” or “effective dose” or “dose” are interchangeably used herein and denote an amount of the pharmaceutical compound having a prophylactically or therapeutically relevant effect on a disease or pathological conditions, i.e. which causes in a tissue, system, animal or human a biological or medical response which is sought or desired, for example, by a researcher or physician.

The aforementioned medical products of the inventive use are particularly used for the therapeutic treatment. Monitoring is considered as a kind of treatment, wherein the compounds are preferably administered in distinct intervals, e.g. in order to booster the response and eradicate the pathogens and/or symptoms of the estrogen-related disease completely. Either the identical compound or different compounds can be applied. The medicament can also be used to reducing the likelihood of developing a disease or even prevent the initiation of diseases associated with estrogen receptor signaling in advance or to treat the arising and continuing symptoms. In the meaning of the invention, prophylactic treatment is advisable if the subject possesses any preconditions for the aforementioned physiological or pathological conditions, such as a familial disposition, a genetic defect, or a previously passed disease.

The diseases as concerned by the invention are preferably cancer (particularly breast cancer, colon cancer and uterine endometrial adenocarcinoma), Alzheimer's disease, cataracts, shock (particularly maintaining vascular volume in septic shock), menopausal symptoms such as post-menopausal calcium deficiencies (particularly inadequate calcium uptake and osteoporosis in postmenopausal women), cardiovascular diseases and conditions of decreased renal blood flow (particularly those caused by diuretics or congestive heart failure). Further conditions associated with estrogen regulation of gene expression in the kidney are known in women, wherein high estrogen levels preceding ovulation during pregnancy and resulting from estrogen administration commonly results in body water retention. Increased renal sodium reabsorption is also a major mechanistic component for the elevated fluid retention. Estrogen has been shown to increase thiazide-sensitive NaCl cotransporter expression levels, providing one possible molecular basis for estrogen effects on sodium retention. Further preferred biological conditions in the meaning of the present invention include inflammation, diabetes, prostate health, abnormal cell development and infectious diseases (see WO 2003/040404 A1).

The said compounds according to the invention can be used in their final non-salt form. On the other hand, the present invention also encompasses the use of these compounds in the form of their pharmaceutically acceptable salts, which can be derived from various organic and inorganic acids and bases by procedures known in the art. The expressions “pharmaceutically acceptable salt” and “physiologically acceptable salt”, which are used interchangeable herein, in the present connection are taken to mean an active ingredient which comprises a compound according to the invention in the form of one of its salts, in particular if this salt form imparts improved pharmacokinetic properties on the active ingredient compared with the free form of the active ingredient or any other salt form of the active ingredient used earlier. The pharmaceutically acceptable salt form of the active ingredient can also provide this active ingredient for the first time with a desired pharmacokinetic property which it did not have earlier and can even have a positive influence on the pharmacodynamics of this active ingredient with respect to its therapeutic efficacy in the body.

Object of the invention is also the use of at least one gene of Table 1 as marker gene for screening compounds with estrogenic or anti-estrogenic activity. Another object of the invention is also the use of multiple genes of Table 1 and optionally Table 4 as marker genes for characterizing estrogenicity compound-specifically. The prior teaching of the present specification concerning the screening method is valid and applicable without restrictions to said uses if expedient.

It is still another object of the present invention to use substances specifically interacting with at least one gene product encoded by a gene of table 1 for detecting estrogenic or anti-estrogenic activity. The term “specific substances” as used herein comprises molecules with high affinity to at least one gene product encoded by the selected genes, in order to ensure a reliable binding. The substances are preferably specific to parts of the gene product. Such parts represent a restriction to those regions which are sufficient for the expression of a specific function, i.e. the provision of a structural determinant for recognition. All truncations are inevitably limited by the requirement of preserving the unique recognition. However, the parts of the gene products can be very small. Preferably, the substances are mono-specific in order to guarantee an exclusive and directed interaction with the chosen single target.

The recognition of the gene product or parts thereof according to the invention can be realized by a specific interaction with substances on the primary, secondary and/or tertiary structure level of a nucleic acid sequence bearing the gene sequence or an amino acid sequence expressed by the gene. The coding function of genetic information favors the primary structure recognition, Contrary to that, the three-dimensional structure is mainly to be considered for protein recognition. In the context of the present invention, the term “recognition”—without being limited thereto—relates to any type of interaction between the specific substances and the target, particularly covalent or non-covalent binding or association, such as a covalent bond, hydrophobic/hydrophilic interactions, van der Waals forces, ion pairs, hydrogen bonds, ligand-receptor interactions, interactions between epitope and antibody binding site, nucleotide base pairing, and the like. Such association may also encompass the presence of other molecules such as peptides, proteins or other nucleotide sequences.

The specific substances are composed of biological and/or chemical structures capable to interact with the target molecule in such a manner that makes a recognition, binding and interaction possible. In particular, the substances are selected from the group of nucleic acids, peptides, carbohydrates, polymers, small molecules having a molecular weight between 50 and 1.000 Da and proteins, preferably nucleic acids. The specific substances express a sufficient sensitivity and specificity in order to ensure a reliable detection.

The proteins or peptides are preferably selected from the group consisting of antibodies, cytokines, lipocalins, receptors, lectins, avidins, lipoproteins, glycoproteins, oligopeptides, peptide ligands and peptide hormones. More preferably, antibodies are used as specific substance. “Antibody” denotes a polypeptide essentially encoded by an immunoglobulin gene or fragments thereof. According to the invention, antibodies are present as intact immunoglobulins or a number of well-characterized fragments. Fragments are preferably selected from the group consisting of F_{ab} fragments, F_{c} fragments, single chain antibodies (scFv), variable regions, constant regions, H chain (V_{H}), and L chain (V_{L}) more preferably F_{ab} fragments and scFv. Fragments, such as F_{ab} fragments and F_{c} fragments, can be produced by cleavage using various peptidases. Furthermore, fragments can be engineered and recombinantly expressed, preferably scFv.

The term “nucleic acid” refers to a natural or synthetic polymer of single- or double-stranded DNA or RNA alternatively including synthetic, non-natural or modified nucleotides, which can be incorporated in DNA or RNA polymers. Each nucleotide consists of a sugar moiety, a phosphate moiety, and either a purine or pyrimidine residue. The nucleic acids are preferably single or double stranded DNA or RNA, primers, antisense oligonucleotides, ribozymes, DNA enzymes, aptamers and/or siRNA, or parts thereof. The nucleic acids can be optionally modified as phosphorothioate DNA, locked nucleic acid (LNA), peptide nucleic acid (PNA) or spiegelmer.

A “nucleic acid probe” is a nucleic acid capable of binding to a target nucleic acid or complementary sequence through one or more types of chemical bond, usually through complementary base pairing by hydrogen bond formation. As used herein, a probe may include natural (i.e. A, G, C, or T) or modified bases (e.g. 7-deazaguanosine, inosine, etc.). In addition, the bases in a probe may be joined by a linkage other than a phosphodiester bond, so long as it does not interfere with hybridization. It will be understood by one of skill in the art that probes may bind target sequences that lack complete complementarity with the probe sequence depending upon the stringency of the hybridization conditions. The probes are preferably directly labeled with isotopes, e.g. chromophores, luminphores or chromogens, or indirectly labeled with biotin to which a streptavidin complex may later bind. By assaying the presence or absence of the probe, one can detect the presence or absence of a target gene of interest.

Particular preferred nucleic acid probes to be used as estrogenicity-specific substances are oligonucleotide probes.

The specific substances can be labeled, in doing so the labeling depends on their inherent features and the detection method to be applied. For the detection of the specific incubation products, the applied methods depend on the specific incubation products to be monitored and are well known to the skilled artisan. Examples of suitable detection methods according to the present invention are fluorescence, luminescence, VIS coloring, radioactive emission, electrochemical processes, magnetism or mass spectrometry.

A labeling method is not particularly limited as long as a label is easily detected. A “labeled nucleic acid or oligonucleotide probe” is one that is bound, either covalently through a linker or a chemical bond, or noncovalently through ionic, van der Waals, electrostatic, hydrophobic interactions or hydrogen bonds, to a label such that the presence of the nucleic acid or probe may be detected by detecting the presence of the label bound to the nucleic acid or probe. In a preferred embodiment of the present invention, the nucleic acids are labeled with digoxigenin, biotin, chemiluminescence substances, fluorescence dyes, magnetic beads, metallic beads, colloidal particles, electron-dense reagents, enzymes, all of them are well-known in the art, or radioactive isotopes. Preferred isotopes for labeling nucleic acids in the scope of the invention are ³H, ¹⁴C, ³²P, ³³P, ³⁵S, or ¹²⁵I, more preferred ³²P, ³³P, or ¹²⁵I.

Further, the invention may be practiced as a kit comprising substances specifically interacting with at least one gene product encoded by a gene of Table 1, particularly in order to perform the inventive method for detecting and/or characterizing estrogenic or anti-estrogenic activity. The kit of the invention may include an article that comprises written instructions or directs the user to written instructions for how to practice the method of the invention. In an embodiment, the kit further comprises a reporter moiety or a reporter apparatus, preferably a fluorophore or a field-effect transistor. Additionally, the kit may comprise an extracting reagent for isolating a nucleic acid, preferably an mRNA. The prior teaching of the present specification concerning the screening method is considered as valid and applicable without restrictions to the kit if expedient.

Yet another object of the invention relates to a gene chip comprising any one or more of the plurality of genes according to any Table 1 to 12 or combinations thereof.

In the scope of the present invention, a method for screening compounds with estrogenic activity, which applies unique gene expression patterns of at least one gene selected from the group comprising the genes of Table 1, is provided for the first time. The present invention teaches characteristic expression fingerprints and a subset of marker genes that are associated with estrogenicity, and it implies that the genes are involved in endocrine disrupting effects. The global gene expression patterns as described herein have been induced by known EACs in ER-positive Ishikawa plus but not in ER-negative Ishikawa minus human endometrial carcinoma cells using Illumina's bead based microarray platform. Diethylstilbestrol (DES), Resveratrol (RESV), Genistein (GEN), Zearalenone (ZEA), Bisphenol A (BPA), and o,p′-Dichlordiphenyl-trichloethane (DDT) have been used as test compounds and (co-)treatment with the anti-estrogen ICI 182,780 (ICI) has been performed to assess ER-dependent responses. The analysis of the differential expressed genes is very suitable for large-scale screening tests. In doing so, chemicals can be identified with an unknown mode of action and predicting their potential to exert endocrine disrupting effects. The detection method as well as arising monitoring method of the invention can be performed in a simple and fast manner. In addition, the appropriate kit is cost-efficiently produced. The characterization of 87 genes of Table 1 and Table 4, particularly 49 genes of Table 7, critically involved in estrogenicity also resulted in the provision of pharmaceutical compositions for the diagnosis, prophylactic or therapeutic treatment and/or monitoring of conditions, which are caused, mediated and/or propagated by estrogen receptor signaling. Their use is a promising, novel approach for a broad spectrum of therapies causing a direct and immediate reduction of symptoms that are clearly connected with estrogen-dependent diseases. In in-vitro screening and monitoring such physiological or pathological conditions, the genes of Table 1 are qualified as biomarkers for detecting and characterizing estrogenicity. Targeting gene products encoded by the genes of Table 1 is highly specific for the estrogenic activity and driven medical disorders therefrom. All substances are characterized by a high affinity, specificity and stability; low manufacturing costs and convenient handling. These features form the basis for a reproducible action, wherein the lack of cross-reactivity is included, and for a reliable and safe interaction with their matching target structures.

All the references cited herein (including US 2003/0008309 A1 and U.S. Pat. No. 7,371,207 B2) are incorporated by reference in the disclosure of the invention hereby.

It is to be understood that this invention is not limited to the particular methods, specific substances, uses and kits described herein, as such matter may, of course, vary. It is also to be understood that the terminology used herein is for the purpose of describing particular embodiments only and is not intended to limit the scope of the present invention, which is only defined by the appended claims. As used herein, including the appended claims, singular forms of words such as “a,” “an,” and “the” include their corresponding plural referents unless the context clearly dictates otherwise. Thus, e.g., reference to “a substance” includes a single or several different substances, and reference to “a method” includes reference to equivalent steps and methods known to a person of ordinary skill in the art, and so forth. Unless otherwise defined, all technical and scientific terms used herein have the same meaning as commonly understood by a person of ordinary skill in the art to which this invention belongs.

Although methods and materials similar or equivalent to those described herein can be used in the practice or testing of the present invention, suitable examples are described below. The following examples are provided by way of illustration and not by way of limitation. Within the examples, standard reagents and buffers that are free from contaminating activities (whenever practical) are used. The examples are particularly to be interpreted such that they are not limited to the explicitly demonstrated combinations of features, but the exemplified features may be unrestrictedly combined again if the technical problem of the invention is solved.

The following abbreviations are used herein:

### BPA: Bisphenol A

### DES: Diethylstilbestrol

DDT: o,p′-Dichlordiphenyltrichlorethane
EAC: endocrine active compound
ER: estrogen receptor

### GEN: Genistein

### GO: Gene Ontology

hd: high dose

### ICI: ICI 182,780

Id: low dose

### RESV: Resveratrol

### TLDA: TaqMan® Low Density Arrays

### ZEA: Zearalenone

### Chemicals and Cell Culture Media Supplements

DES (purity≧99%), GEN (purity≧98%), ZEA (purity≧99%), RESV (purity>99%), BPA (purity≧99%) and penicillin/streptomycin solution were purchased from Sigma-Aldrich (Taufkirchen, Germany), o,p′-DDT (purity≧99%) was from Chem Service (West Chester, USA), ICI 182,780 was obtained from Tocris (Ellisville, USA), DMEM/F12, Gentamicin and sodium pyruvate were purchased from Invitrogen Corp. (Karlsruhe, Germany). Foetal bovine serum (FBS) was delivered by Biochrome KG (Berlin, Germany) and dextran-coated charcoal FBS (DCC/FBS) was from Hyclone (Lot AKD11642A, Perbio Science, Bonn, Germany).

### Cell Culture and Dose Selection

Human Ishikawa plus cell line (ECACC Order No. 99040201) was derived from a human endometrial adenoma and expresses endogenous ERα. The ER-deficient Ishikawa minus cell line was obtained from K. Korach (NIEHS, NC, USA) (Ignar-Trowbridge et al. 1993 Mol Endocrinol 7(8): 992-998). Cells were routinely maintained in DMEM/F12 with L-Glutamine and 15 mM Hepes supplemented with 10% (v/v) FBS, 1% (v/v) penicillin (10 kU/ml)-streptomycin (10 mg/ml) solution, 0.1% (v/v) Gentamicin (50 mg/ml) and 1 mM sodium pyruvate at 37° C. and 5% CO₂ in culture flasks. 5×10⁵ cells were seeded onto 6-well plates in phenol-red free DMEM/F12 with L-Glutamine and 15 mM Hepes containing 10% (v/v) dextran-coated charcoal treated FBS, sodium pyruvate and antibiotics. Cells were cultured at 37° C. and 5% CO₂ for 24 h prior to treatment with either 0.5% (v/v) ethanol as vehicle control or compounds at two dose levels, which were selected from ERα transactivation studies using a consensus ERE as a reporter (Mueller et al. 2004 Toxicol Sci 80 (1): 14-25).

The low dose (Id) level corresponds to the compound concentration causing a half maximal activation of the reporter gene (EC₅₀), whereas the high dose (hd) refers to the dose at saturated activity. The following doses were chosen for the experiments: DES 0.05 nM (Id)/1 nM (hd), GEN 200 nM (Id)/1 μM (hd), ZEA 2.5 nM (Id)/100 nM (hd), RESV 7.5 μM (Id)/50 μM (hd), BPA 2 μM (Id)/5 μM (hd), o,p′-DDT 4 μM (Id)/10 μM (hd). Cells were also treated with the anti-estrogen ICI 182,780 (100 nM) and in combination with the test compounds at high doses. All experiments were performed three times with cells of passages 3-25.

### RNA Extraction

After a 24 h treatment period cells were rinsed with PBS (Gibco Invitrogen, Karlsruhe, Germany), harvested and total RNA was extracted using the RNeasy Mini Kit (QIAGEN, Hilden, Germany) as described by the manufacturer, including the QIAshredder spin column procedure and on-column RNase-free DNase digestion. RNA was eluted with 40 μl of RNA-Storage solution (Ambion, Darmstadt, Germany). RNA quality control and quantification was determined using a NanoDrop® spectrophotometer (Kisker, Steinfurt, Germany) and 2100 Agilent Bio-Analyzer (Agilent Technologies, Waldbronn, Germany). Only RNA with a quality ratio A260/A280 between 1.9 and 2.1 and no evidence of peak degradation (18s/28s) was used.

cRNA Synthesis and Illumina Whole Genome Chip Hybridization

Gene expression analysis was executed using Illumina Sentrix® HumanRef-8 V2 BeadChip Arrays (Illumina Inc., San Diego, Calif., USA) allowing the analysis of ˜23,000 transcripts. Synthesis of biotin-labeled cRNA was performed in an automated procedure using a Theonyx Liquid Performer (Aviso GmbH, Greiz, Germany) and MessageAmp™ II aRNA amplification Kit (Ambion, Darmstadt, Germany) with several modifications requested by Illumina to optimize the process (Zidek et al. 2007 Toxicol Sci 99(1): 289-302). Instead of column cleanup, the bead-based Agencourt® RNAcIean™ system (Beckman Coulter, Krefeld, Germany) was applied to purify cDNA and cRNA. cRNA quantity was measured spectrophotometrically (NanoDrop®) and the 2100 Agilent Bio-Analyzer was used for quality assessment.

Seven hundred fifty nanograms of amplified biotinylated cRNA were hybridized onto the Illumina Sentrix® BeadChip in a Hybridization Cartridge under humidified conditions for 20 h at 58° C. (Hybridization oven, Illumina Inc., San Diego, Calif., USA). The chips were then washed, stained for 10 minutes with 1 μg/ml streptavidin-conjugated Cy3 (Amersham Biosciences, Buckinghamshire, UK) and finally dried by centrifugation according to the protocol provided. Fluorescence detection was carried out by confocal laser scanning with the Illumina® BeadArray Reader (Illumina, Inc., San Diego, Calif., USA) at 532 nm and 0.8 μm resolution.

### Statistical Data Analysis

The array data analysis is a multi-step process beginning with decoding the image spots because of random assembly of the microbeads on the array surface (Gunderson et al. 2004 Genome Res 14 (5): 870-877; Kuhn et al. 2004 Genome Research 14: 2347-2356). Each bead type is represented on average 30 times per array providing internal replicates (Steemers and Gunderson 2005 Pharmacogenomics 6 (7): 777-782). IIlumina® BeadStudio Software was used for condensing these data and further to ensure array quality based on different control bead parameters.

Data were uploaded into Genedata's Expressionist® Analyst software (Genedata AG, Basel, Switzerland) for data normalization and statistical analysis. Data were normalized using Lowess (Locally Weighted Linear Regression) to offset non-biological differences (systematic variation) between the samples and arrays. After normalization fold-regulations for each individual compound treatment were calculated. The global gene set was reduced by filtering any signal intensity<200 (background level) to reduce error rates of multiple testing during further analysis.

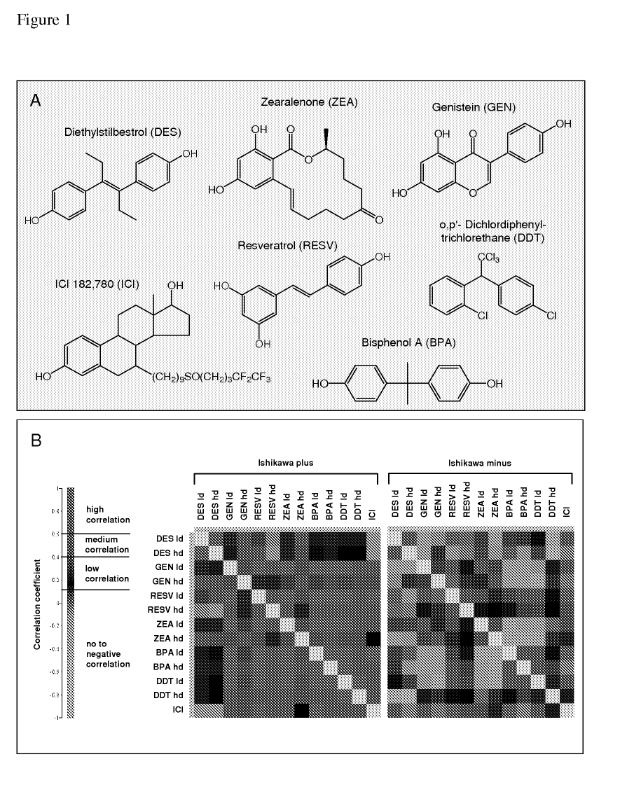

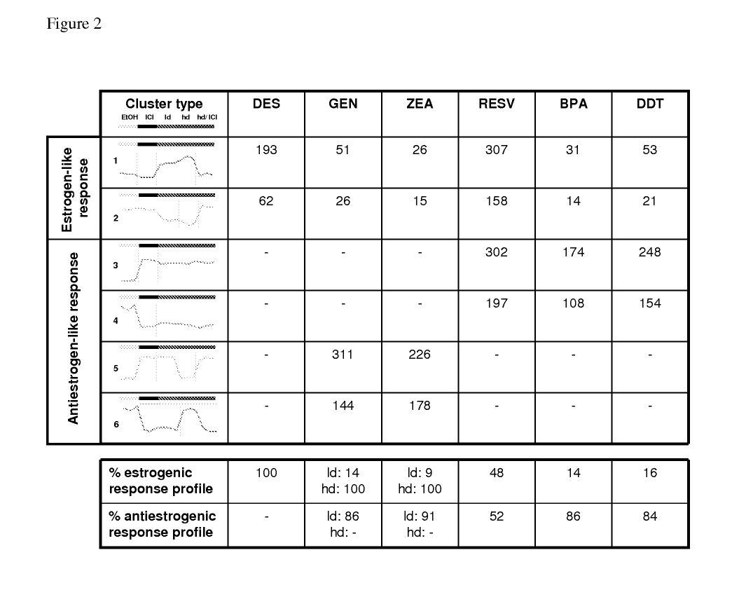

For investigation of compound-specific differentially expressed genes and to select genes that showed an ER-dependent regulation, gene expression profiles of each compound were compared individually and in combination with the anti-estrogenic ICI against the vehicle control by Analysis of Variance (ANOVA). BH-Q-values (Benjamini and Hochberg false discovery rate) were used as a significance measure in addition to the p-value (Benjamini and Hochberg 1995 J R Statist Soc 57 (1): 289-300). Thus, only genes with a p-value and BH-Q-value less than 0.01 were accepted as significant. Genedata's Expressionist® unsupervised K-Means cluster algorithm was used to group the selected genes into randomly determined different partitions (cluster), facilitating identification of (anti-)estrogen-like gene expression profiles (FIG. 2). In addition, a cross-compound comparison with significantly deregulated genes (p-value/BH-Q-value<0.01) from each compound was executed by calculating Pearson correlation coefficients for the different treatment experiments (FIG. 1B).

For selection of characteristic estrogenicity marker genes, ANOVA was applied to the tested EACs against the vehicle controls. Only genes having a p-value and BH-Q-value less than 0.01 were accepted as significant. The gene group was further condensed by selecting genes with more than 1.5 fold-regulation in DES hd samples, which improved the selection of potential marker genes.

All data were recorded in compliance with MIAME (Minimum Information About a Microarray Experiment) recommendations.

cDNA Synthesis for TaqMan® Low Density Arrays (TLDA)

For verification of the Illumina results 2 μg of purified total RNA was reverse transcribed to cDNA using random hexamer primers with the Transcriptor First Strand cDNA Synthesis Kit (Roche, Mannheim, Germany) according to the manufacturer's protocol. Quality and quantity was done by mRNA Pico Chip Assay with the 2100 Agilent Bio-Analyzer (Agilent Technologies, Waldbronn, Germany).

### TLDA Preparation and Analysis

Real-time PCR on selected genes and samples was performed using 384-well TaqMan® Low Density Arrays (Applied Biosystems, Darmstadt, Germany) allowing analysis of eight samples in parallel (Tuschl and Mueller 2006 Toxicology 218 (2-3): 205-215). Efficiency values were calculated from a PCR reaction performed with a dilution series of cDNA (500 ng, 50 ng, 5 ng, 0.5 ng, 0.05 ng per sample well in duplicate) prepared from Ishikawa plus total RNA. Gene expression analysis was done by the efficiency-corrected ΔΔCt method from Livak and Schmittgen, determining target gene expression relative to 18S rRNA (housekeeping control) and relative to the vehicle control (Livak and Schmittgen 2001 Methods 25(4): 402-408). T-Test was performed for significance testing using Genedata's Expressionist® Analyst software, whereby only genes having a p-value less than 0.05 were accepted as significant.

FIG. 1

(A) Structures of herbal, fungal and synthetic compounds with potential endocrine activity. (B) Experimental correlation analysis on significantly regulated genes from Illumina global gene expression profiling (p-value/BH-q-value<0.01) in Ishikawa plus and Ishikawa minus cells treated with low dose (Id) and high dose (hd) of DES, GEN, RESV, ZEA, BPA, DDT and/or ICI for 24 h. Calculation of correlation distances and imaging was done by Genedata's Expressionst® Analyst software (Genedata AG, Basel, Switzerland). The magnitude of Pearson's correlation coefficients is indicated by the color scale and corresponding annotations.

FIG. 2

K-Means cluster analysis of gene expression and number of deregulated genes in Ishikawa plus cells after treatment with various EACs. K-Means cluster of significantly regulated genes (p-value/BH-Q-value<0.01) were compared after treatment with EACs at low dose (Id) and high dose (hd), anti-estrogenic ICI or combination treatment of EAC hd level and ICI. Clusters 1 and 2 contain genes with diametrical expression profiles between EACs and anti-estrogenic ICI treatments. Therefore, these genes may be regulated by classical estrogen receptor-dependent signaling and are referred to as estrogen-dependent gene clusters. Genes displaying an expression pattern in response to EACs similar to ICI are summarized in clusters 3-6. Clusters 3 and 4 comprised genes exerting these profiles at both EAC dose treatments, whereas clusters 5 and 6 genes displayed anti-estrogenic effects only at Id treatments. The proportion of estrogen-like and anti-estrogen-like gene regulation (% estrogenic/anti-estrogenic response profile) was calculated against the total number of significantly regulated genes for each compound.

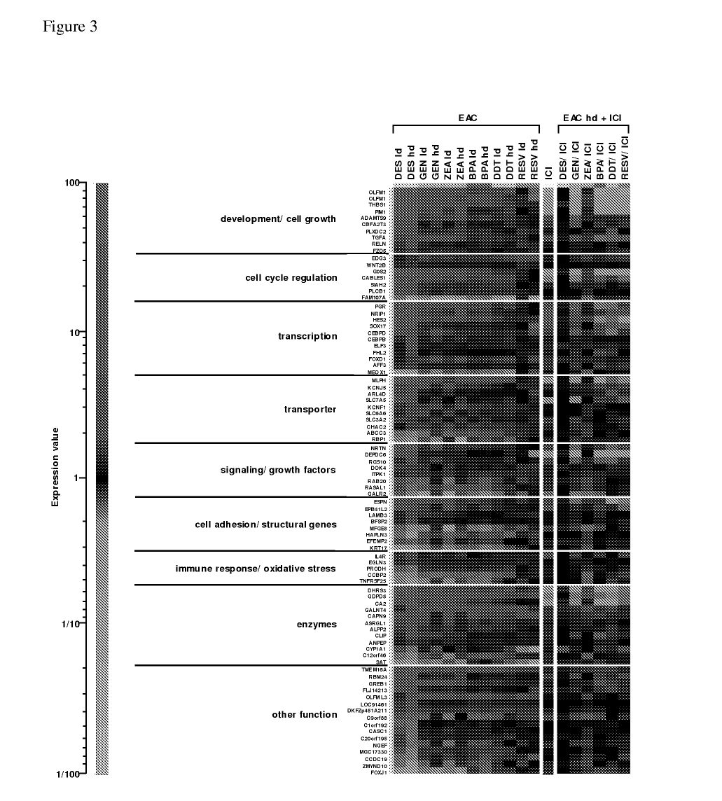

FIG. 3

Gene profile display of differentially regulated genes in Ishikawa plus cells after treatment with various EACs at low dose (Id) and high dose (hd) and/or ICI for 24 h. Responses to EACs were analyzed using Illumina arrays. The 61 up- and 26 down-regulated genes, which are shown in the heatmap, were found to be significantly regulated (p-value/BH-q-value<0.01) and having a fold-regulation>1.5 for DES hd treatment. The color scale corresponds to fold-change in gene expression: stimulated genes are shown in red, inhibited genes in green, and genes not regulated in black. Abbreviations of marker genes are given as supplementary information.

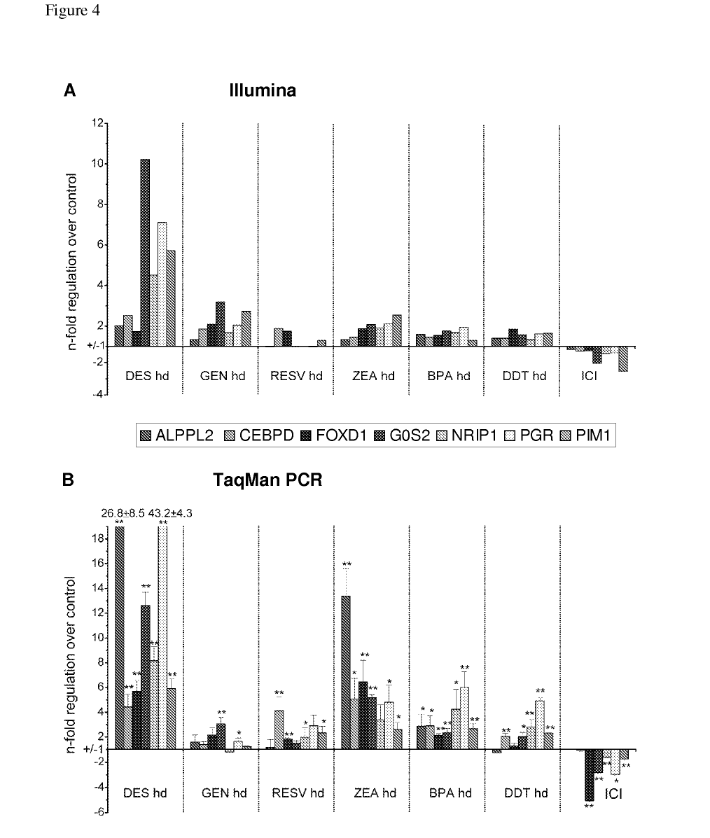

FIG. 4

Selected genes from Illumina experiments in Ishikawa plus cells were subjected to TaqMan PCR to verify the microarray results. Data were analyzed by ΔΔCt method relative to 18s housekeeper gene expression. Fold-regulations were calculated for both techniques against vehicle-control. *p<0.05, **p<0.01 (student's t-test).

Table 1 lists novel estrogen/estrogen receptor target genes differentially expressed after EAC exposure.

Table 2 lists novel estrogen/estrogen receptor target genes that are up-regulated after EAC exposure.

Table 3 lists novel estrogen/estrogen receptor target genes that are down-regulated after EAC exposure.

Table 4 lists known estrogen/estrogen receptor target genes differentially expressed after EAC exposure.

Table 5 lists known estrogen/estrogen receptor target genes that are up-regulated after EAC exposure.

Table 6 lists novel estrogen/estrogen receptor target genes that are down-regulated after EAC exposure.

Table 7 lists novel estrogen/estrogen receptor target genes differentially expressed after EAC exposure (subset of Table 1).

Table 8 lists novel estrogen/estrogen receptor target genes that are up-regulated after EAC exposure (subset of Table 2).

Table 9 lists novel estrogen/estrogen receptor target genes that are down-regulated after EAC exposure (subset of Table 3).

Table 10 lists genes of estrogen signaling differentially expressed after EAC exposure.

Table 11 lists genes of estrogen signaling that are up-regulated after EAC exposure.

Table 12 lists genes of estrogen signaling that are down-regulated after EAC exposure.

### EXAMPLE 1

### EACs Induce Characteristic Gene Expression Patterns in Ishikawa Plus but not in Ishikawa Minus Cells

The main focus was to investigate gene expression patterns of chemicals known or suspected to have estrogenic activity. DES served as a reference compound. RESV and GEN, two representatives of phytoestrogens, the mycotoxin ZEA as well as the chemicals BPA and o,p′-DDT (FIG. 1A) were chosen for the analysis of molecular responses in Ishikawa cells by measuring genome wide transcript level changes. Additional treatment with the pure anti-estrogen ICI, either alone or in combination with EACs, was included to elucidate ER-dependent responses and discriminate common non-specific changes resulting from cellular stress. The majority of estrogenic effects at the molecular level are mediated by estrogen receptors, prompting to choose the ERα-positive Ishikawa plus cells for the experiments and compare the results to ER-negative Ishikawa minus cell line that is not estrogen responsive.

Correlation analysis were performed on a set of 1682 (Ishikawa plus) and 1265 (Ishikawa minus) genes, created by addition of all significantly deregulated genes selected by ANOVA for each compound treatment (FIG. 1B). Correlation results were used to estimate (dis-)similarities to DES reference, as well as to the anti-estrogen ICI. ICI competes with other ER ligands for the ligand binding site and thus can inhibit downstream signal transduction events. Therefore, similarities in the gene expression profile between the tested compounds and ICI indicate anti-estrogenic gene regulation.

Analysis of gene expression in Ishikawa plus cells revealed a low positive correlation for BPA, DDT and low doses of GEN and ZEA compared with DES, whereas high dose GEN and ZEA displayed medium to high correlations. In contrast, no correlation was found between RESV treatment and DES. Interestingly, RESV showed a high positive correlation to ICI, which was similar for BPA, DDT, and low doses of GEN and ZEA. In accordance with the high correlation between GEN and ZEA high doses to DES, little similarity was observed with ICI. DES induced expression was negatively correlated to that of ICI. Taken together, cross-compound comparison showed highly similar gene expression profiles for GEN and ZEA low doses, BPA, DDT, and RESV as well as for DES compared to high dose treatments of GEN and ZEA (FIG. 1B).

In contrast to the results in Ishikawa plus, the gene expression patterns in the ER-deficient Ishikawa minus were random and without noticeable correlation tendencies between the EACs and ICI (FIG. 1B). In fact, only few significantly deregulated genes and weaker fold-changes were observed in Ishikawa minus. This further proved that ER is the major regulator of transcriptional effects of the tested EACs.

### EXAMPLE 2

### Cluster Analyses Revealed (Anti-)Estrogenic Properties of EACs in Ishikawa Plus Cells

K-Means clustering with Ishikawa plus cells leads to a more detailed insight into the gene expression pattern and potential effect of ICI on significantly regulated genes (FIG. 2). K-Means clustering has the power to group genes with similar expression profiles together and separate them from other dissimilar gene groups. It was focused on identifying genes whose transcription can be regulated by estrogen receptors. Clusters 1 and 2 represent such potential ER-dependent regulated genes, because treatment with ICI or a combination of ICI/compound at high dose displayed a weaker or opposite regulation compared to treatment with the compound alone. While DES analysis revealed only genes with ER-dependent expression profiles (193 up- and 62 down-regulated), further clusters were discovered for the other test compounds, suggesting a distinct mode of action. The inventors found a set of genes similarly regulated by ICI and low dose treatments of GEN and ZEA (clusters 5 and 6), amounting approximately 86% of GEN and 91% of ZEA low dose deregulated genes. Only a minority of genes seemed to be regulated in an ER-dependent manner. In contrast, at high dose levels of GEN and ZEA, only ER-dependent regulated genes were identified. Thus, GEN and ZEA showed distinct expression patterns at low versus high doses indicating a bi-phasic dose-response relationship of EACs. Approximately 66% of these ER-dependent genes regulated by GEN or ZEA were also deregulated in the same direction after DES treatment, indicating DES-like estrogenic activity of GEN and ZEA. Moreover, the anti-estrogenic portion of the gene expression patterns of low doses of GEN and ZEA were compared. Both showed similar expression of more than 50% of anti-estrogen-like up- and down-regulated genes (clusters 5 and 6).

K-Means analysis of significantly regulated genes after RESV, BPA and DDT treatments resulted in four different cluster types (FIG. 2). Clusters 1 and 2 (ER-dependent regulated genes) and two other specific gene groups, comprising genes up-(cluster 3) or down-regulated (cluster 4) in response to treatment with compound, ICI or both. Therefore, clusters 3 and 4 indicate an anti-estrogenic, ICI-like, expression pattern and include the majority of significantly regulated genes by RESV, BPA, and DDT. For RESV treatment, both estrogen-like (48%) and anti-estrogen-like (52%) gene expression was observed. Despite several deregulations within the putative estrogenic response, only 6% of these genes were also found to be unidirectionally regulated after DES treatment. These results suggest that RESV may induce considerable changes in the expression pattern of ER-dependent regulated genes, but in a different manner from DES. The expression changes induced by BPA and DDT were predominately characterized by anti-estrogen-like gene regulations corresponding to 86% and 85% of significantly regulated genes by BPA and DDT, respectively. However, although a fewer number of genes were potentially ER-dependently regulated, the concordance to DES was considerable. Eighty percent of BPA and 72% of DDT regulated cluster 1 and 2 genes were also differentially expressed in the same direction after DES exposure. Furthermore, many ICI-like regulated genes (clusters 3 and 4) of BPA and DDT were found to be unidirectionally regulated by RESV. The same was true after low dose GEN and ZEA treatments (clusters 5 and 6), suggesting a high similarity in anti-estrogenic responses elicited by all these EACs.

Overall, the findings support the results from the correlation analysis, as high correlations were calculated for BPA, DDT, low dose of ZEA and GEN compared with ICI, indicating stronger anti-estrogen-like rather than estrogenic activity. Only genes with ER-dependent regulation profiles were found in response to DES, high dose ZEA and GEN, explaining the excellent correlation between these treatment conditions.

### EXAMPLE 3

### Prospective Candidate Genes for Screening EACs In-Vitro

Global gene expression profiling was used to identify similarly expressed genes, which could serve as putative markers for estrogenic activity. Although these six compounds altered the expression of the genes in Ishikawa plus with different magnitudes, 87 genes (61 up- and 26 down-regulated) were found to be similarly regulated across all compounds (FIG. 3), indicating similar estrogenic mechanisms of action with exception of RESV discussed more detailed later. Most genes showed a weaker regulation score or were diametrically regulated by ICI and ICI/compound combination treatments, proving the ER-dependent regulation of these genes. Functional annotation of these marker genes revealed genes related to cell proliferation and differentiation, transcriptional regulation, immune response, cell signaling and intracellular transport.

For verification of our Illumina results, expression levels of the selected estrogenic marker genes, ALPP2, CEBPD, FOXD1, G0S2, NRIP1, PGR and PIM1 were quantified by real-time PCR (FIG. 4). Although gene expression was altered with different magnitudes, the real-time PCR expression patterns confirmed IIlumina gene expression results.

In the evaluation of EACs, specific similarities in the expression patterns to the anti-estrogenic compound ICI were also observed. BPA, DDT, ZEA and GEN showed a good correlation to ICI, supporting the suggested anti-estrogenic properties of these compounds. In contrast, RESV not only regulates 52% of its significantly regulated genes in the same direction as ICI, it was also shown to regulate one third of the putative estrogenic marker genes unidirectional to ICI. This effect was even more pronounced at high doses of RESV. Thus, this particular mixed estrogenic/anti-estrogenic activity pattern may explain the suggested beneficial effects of these compounds as RESV and GEN exert cancer preventive and cardioprotective effects. However, the data indicated that dose plays a key role, as seen for GEN and ZEA, which displayed anti-estrogenic gene regulation only at low doses.

TABLE 1

| Name | Accession | Symbol | Definition | Synonym |
|---|---|---|---|---|
| ILMN_4188 | NM_003857.2 | GALR2 | Homo sapiens galanin receptor 2 (GALR2), | GALNR2 |
| | | | mRNA. | |
| ILMN_5566 | NM_000422.1 | KRT17 | Homo sapiens keratin 17 (KRT17), mRNA. | PC; K17; PC2; PCHC1 |
| ILMN_11289 | NM_002899.2 | RBP1 | Homo sapiens retinol binding protein 1, cellular | CRBP; RBPC; CRBP1; |
| | | | (RBP1), mRNA. | CRABP-I |
| ILMN_12497 | NM_002970.1 | SAT | Homo sapiens spermidine/spermine N1- | DC21; KFSD; SSAT |
| | | | acetyltransferase (SAT), mRNA. | |
| ILMN_26083 | NM_152321.1 | C12orf46 | Homo sapiens chromosome 12 open reading | FLJ32115 |
| | | | frame 46 (C12orf46), mRNA. | |
| ILMN_29699 | NM_001013625.2 | C1orf192 | Homo sapiens chromosome 1 open reading | — |
| | | | frame 192 | |
| ILMN_7222 | NM_024059.2 | C20orf195 | Homo sapiens chromosome 20 open reading | — |
| | | | frame 195 (C20orf195), mRNA. | |
| ILMN_11032 | NM_022833.2 | C9orf88 | Homo sapiens chromosome 9 open reading | — |
| | | | frame 88 | |
| ILMN_16401 | NM_018272.2 | CASC1 | Homo sapiens cancer susceptibility candidate 1 | — |
| | | | (CASC1), mRNA. | |
| ILMN_11666 | NM_001296.3 | CCBP2 | Homo sapiens chemokine binding protein 2 | D6; hD6; CCR9; CCR10; |
| | | | (CCBP2), mRNA. | CMKBR9 |
| ILMN_138202 | NM_012337.1 | CCDC19 | Homo sapiens coiled-coil domain containing 19 | — |
| | | | (CCDC19), mRNA. | |
| ILMN_137851 | NM_016938.1 | EFEMP2 | Homo sapiens EGF-containing fibulin-like | UPH1; FBLN4 |
| | | | extracellular matrix protein 2 (EFEMP2), mRNA. | |
| ILMN_18028 | NM_007177.1 | FAM107A | Homo sapiens family with sequence similarity | DRR1; TU3A |
| | | | 107, member A (FAM107A), mRNA. | |
| ILMN_13823 | NM_001454.2 | FOXJ1 | Homo sapiens forkhead box J1 (FOXJ1), mRNA. | — |
| ILMN_19816 | NM_178232.2 | HAPLN3 | Homo sapiens hyaluronan and proteoglycan link | EXLD1; HsT19883 |
| | | | protein 3 (HAPLN3), mRNA. | |
| ILMN_137320 | NM_004527.2 | MEOX1 | Homo sapiens mesenchyme homeo box 1 | — |
| | | | (MEOX1), transcript variant 1, mRNA. | |
| ILMN_11368 | NM_005928.1 | MFGE8 | Homo sapiens milk fat globule-EGF factor 8 | BA46; EDIL1; OAcGD3S; |
| | | | protein (MFGE8), mRNA. | HsT19888 |
| ILMN_15026 | NM_052880.3 | MGC17330 | Homo sapiens HGFL gene (MGC17330), mRNA. | — |
| ILMN_26971 | NM_019850.1 | NGEF | Homo sapiens neuronal guanine nucleotide | EPHEXIN |
| | | | exchange factor (NGEF), mRNA. | |
| ILMN_17486 | NM_016335.2 | PRODH | Homo sapiens proline dehydrogenase (oxidase) | PIG6; SCZD4; HSPOX2; |
| | | | 1 (PRODH), nuclear gene encoding | PRODH1; PRODH2; |
| | | | mitochondrial protein, mRNA. | TP53I6 |
| ILMN_13192 | NM_017817.1 | RAB20 | Homo sapiens RAB20, member RAS oncogene | — |
| | | | family (RAB20), mRNA. | |
| ILMN_23427 | NM_004658.1 | RASAL1 | Homo sapiens RAS protein activator like 1 | RASAL |
| | | | (GAP1 like) (RASAL1), mRNA. | |
| ILMN_14916 | NM_148973.1 | TNFRSF25 | Homo sapiens tumor necrosis factor receptor | DR3; TR3; DDR3; LARD; |
| | | | superfamily, member 25 (TNFRSF25), transcript | APO-3; TRAMP; WSL-1; |
| | | | variant 10, mRNA. | WSL-LR; TNFRSF12 |
| ILMN_23986 | NM_015896.2 | ZMYND10 | Homo sapiens zinc finger, MYND-type | — |
| | | | containing 10 (ZMYND10), mRNA. | |
| ILMN_20088 | NM_001150.1 | ANPEP | Homo sapiens alanyl (membrane) | CD13; LAP1; PEPN; gp150 |
| | | | aminopeptidase (aminopeptidase N, | |
| | | | aminopeptidase M, microsomal aminopeptidase, | |
| | | | CD13, p150) (ANPEP), mRNA. | |
| ILMN_26976 | NM_138375.1 | CABLES1 | Homo sapiens Cdk5 and Abl enzyme substrate 1 | HsT2563; FLJ35924 |
| | | | (CABLES1), mRNA. | |
| ILMN_24376 | NM_022783.1 | DEPDC6 | Homo sapiens DEP domain containing 6 | DEP.6; FLJ12428; |
| | | | (DEPDC6), mRNA. | FLJ13854; |
| | | | | DKFZp564B1778 |
| ILMN_17789 | NM_000418.2 | IL4R | Homo sapiens interleukin 4 receptor (IL4R), | CD124; IL4RA |
| | | | transcript variant 1, mRNA. | |
| ILMN_3945 | NM_182734.1 | PLCB1 | Homo sapiens phospholipase C, beta 1 | PLC-I; PI-PLC; PLC-154 |
| | | | (phosphoinositide-specific) (PLCB1), transcript | |
| | | | variant 2, mRNA. | |
| ILMN_27252 | NM_002925.3 | RGS10 | Homo sapiens regulator of G-protein signalling | Impact of Estrogen |
| | | | 10 (RGS10), transcript variant 2, mRNA. | Receptor on Gene |
| | | | | Networks Regulated by |
| | | | | Estrogen . . . Chang 2006 |
| ILMN_7086 | NM_001012661.1 | SLC3A2 | Homo sapiens solute carrier family 3 (activators | 4F2; CD98; MDU1; 4F2HC; |
| | | | of dibasic and neutral amino acid transport), | 4T2HC; NACAE; CD98HC |
| | | | member 2 (SLC3A2), transcript variant 1, | |
| | | | mRNA. | |
| ILMN_25446 | NM_003486.5 | SLC7A5 | Homo sapiens solute carrier family 7 (cationic | E16; CD98; LAT1; 4F2LC; |
| | | | amino acid transporter, y+ system), member 5 | MPE16; hLAT1; D16S469E |
| | | | (SLC7A5), mRNA. | |
| ILMN_28750 | NM_000067.1 | CA2 | Homo sapiens carbonic anhydrase II (CA2), | CA II; CA-II |
| | | | mRNA. | |
| ILMN_4674 | NM_005194.2 | CEBPB | Homo sapiens CCAAT/enhancer binding protein | LAP; CRP2; TCF5; IL6DBP; |
| | | | (C/EBP), beta (CEBPB), mRNA. | NF-IL6; MGC32080; |
| | | | | C/EBP-beta |
| ILMN_6563 | NM_005195.2 | CEBPD | Homo sapiens CCAAT/enhancer binding protein | CELF; CRP3; C/EBP-delta; |
| | | | (C/EBP), delta (CEBPD), mRNA. | NF-IL6-beta |
| ILMN_13615 | NM_004433.3 | ELF3 | Homo sapiens E74-like factor 3 (ets domain | ERT; ESX; EPR-1; ESE-1 |
| | | | transcription factor, epithelial-specific) (ELF3), | |
| | | | mRNA. | |
| ILMN_7287 | NM_031475.1 | ESPN | Homo sapiens espin (ESPN), mRNA. | DFNB36; LP2654; |
| | | | | DKFZP434A196 |
| ILMN_20831 | NM_001450.3 | FHL2 | Homo sapiens four and a half LIM domains 2 | DRAL; AAG11; SLIM3 |
| | | | (FHL2), transcript variant 1, mRNA. | |
| ILMN_25543 | NM_014216.3 | ITPK1 | Homo sapiens inositol 1,3,4-triphosphate 5/6 | ITRPK1 |
| | | | kinase (ITPK1), mRNA. | |
| ILMN_1092 | NM_000228.2 | LAMB3 | Homo sapiens laminin, beta 3 (LAMB3), | LAMNB1 |
| | | | transcript variant 1, mRNA. | |
| ILMN_20333 | NM_020190.2 | OLFML3 | Homo sapiens olfactomedin-like 3 (OLFML3), | OLF44; HNOEL-iso |
| | | | mRNA. | |
| ILMN_21964 | NM_002648.2 | PIM1 | Homo sapiens pim-1 oncogene (PIM1), mRNA. | PIM |
| ILMN_25998 | NM_002285.2 | AFF3 | Homo sapiens AF4/FMR2 family, member 3 | LAF4; MLLT2-like |
| | | | (AFF3), transcript variant 1, mRNA. | |
| ILMN_138313 | NM_001661.2 | ARL4D | Homo sapiens ADP-ribosylation factor-like 4D | ARL6; ARF4L |
| | | | (ARL4D), mRNA. | |
| ILMN_2585 | NM_025080.2 | ASRGL1 | Homo sapiens asparaginase like 1 (ASRGL1), | ALP; ALP1; FLJ22316 |
| | | | mRNA. | |
| ILMN_23075 | NM_003571.2 | BFSP2 | Homo sapiens beaded filament structural protein | CP47; CP49; LIFL-L |
| | | | 2, phakinin (BFSP2), mRNA. | |
| ILMN_934 | NM_016452.1 | CAPN9 | Homo sapiens calpain 9 (CAPN9), transcript | GC36; nCL-4 |
| | | | variant 2, mRNA. | |
| ILMN_11754 | NM_005187.4 | CBFA2T3 | Homo sapiens core-binding factor, runt domain, | ETO2; MTG16; MTGR2; |
| | | | alpha subunit 2; translocated to, 3 (CBFA2T3), | ZMYND4 |
| | | | transcript variant 1, mRNA. | |
| ILMN_1763 | NM_001008708.1 | CHAC2 | Homo sapiens ChaC, cation transport regulator- | — |
| | | | like 2 (E. coli) (CHAC2), mRNA. | |
| ILMN_12477 | NM_003613.2 | CILP | Homo sapiens cartilage intermediate layer | HsT18872 |
| | | | protein, nucleotide pyrophosphohydrolase | |
| | | | (CILP), mRNA. | |
| ILMN_12432 | NM_004753.4 | DHRS3 | Homo sapiens dehydrogenase/reductase (SDR | SDR1; Rsdr1; retSDR1 |
| | | | family) member 3 (DHRS3), mRNA. | |
| ILMN_9402 | NM_001003399.1 | DKFZp451A211 | Homo sapiens DKFZp451A211 protein | — |
| | | | (DKFZp451A211), mRNA. | |
| ILMN_5422 | NM_018110.2 | DOK4 | Homo sapiens docking protein 4 (DOK4), | FLJ10488 |
| | | | mRNA. | |
| ILMN_27171 | NM_022073.2 | EGLN3 | Homo sapiens egl nine homolog 3 (C. elegans) | PHD3; HIFPH3; FLJ21620; |
| | | | (EGLN3), mRNA. | MGC125999 |
| ILMN_6138 | NM_001431.1 | EPB41L2 | Homo sapiens erythrocyte membrane protein | 4.1-G; DKFZp781H1755 |
| | | | band 4.1-like 2 (EPB41L2), mRNA. | |
| ILMN_25398 | NM_024841.2 | FLJ14213 | Homo sapiens hypothetical protein FLJ14213 | MGC16218 |
| | | | (FLJ14213), mRNA. | |
| ILMN_138374 | NM_004472.1 | FOXD1 | Homo sapiens forkhead box D1 (FOXD1), | FKHL8; FREAC4 |
| | | | mRNA. | |
| ILMN_26352 | NM_003468.2 | FZD5 | Homo sapiens frizzled homolog 5 (Drosophila) | HFZ5 |
| | | | (FZD5), mRNA. | |
| ILMN_16210 | NM_030792.4 | GDPD5 | Homo sapiens glycerophosphodiester | PP1665 |
| | | | phosphodiesterase domain containing 5 | |
| | | | (GDPD5), mRNA. | |
| ILMN_15240 | NM_019089.3 | HES2 | Homo sapiens hairy and enhancer of split 2 | — |
| | | | (Drosophila) (HES2), mRNA. | |
| ILMN_823 | NM_002236.4 | KCNF1 | Homo sapiens potassium voltage-gated channel, | IK8; kH1; KCNF; KV5.1; |
| | | | subfamily F, member 1 (KCNF1), mRNA. | MGC33316 |
| ILMN_13852 | NM_000890.3 | KCNJ5 | Homo sapiens potassium inwardly-rectifying | CIR; GIRK4; KATP1; |
| | | | channel, subfamily J, member 5 (KCNJ5), | KIR3.4 |
| | | | mRNA. | |
| ILMN_24839 | NM_138370.1 | LOC91461 | Homo sapiens hypothetical protein BC007901 | — |
| | | | (LOC91461), mRNA. | |
| ILMN_29422 | NM_024101.4 | MLPH | Homo sapiens melanophilin (MLPH), mRNA. | In; MELPH; I1Rk3; |
| | | | | MGC2771; SLAC2-A; Slac- |
| | | | | 2a; FLJ12145; I(1)-3Rk; |
| | | | | exophilin-3 |
| ILMN_25657 | NM_004558.2 | NRTN | Homo sapiens neurturin (NRTN), mRNA. | NTN |
| ILMN_29366 | NM_032812.7 | PLXDC2 | Homo sapiens plexin domain containing 2 | TEM7R; FLJ14623 |
| | | | (PLXDC2), mRNA. | |
| ILMN_6412 | NM_153020.1 | RBM24 | Homo sapiens RNA binding motif protein 24 | RNPC6; FLJ30829; |
| | | | (RBM24), mRNA. | FLJ37697; dJ259A10.1 |
| ILMN_9292 | NM_005045.2 | RELN | Homo sapiens reelin (RELN), transcript variant 1, | RL |
| | | | mRNA. | |
| ILMN_25763 | NM_003043.2 | SLC6A6 | Homo sapiens solute carrier family 6 | TAUT; MGC10619 |
| | | | (neurotransmitter transporter, taurine), member 6 | |
| | | | (SLC6A6), mRNA. | |
| ILMN_8034 | NM_022454.2 | SOX17 | Homo sapiens SRY (sex determining region Y)- | FLJ22252 |
| | | | box 17 (SOX17), mRNA. | |
| ILMN_25243 | NM_018043.4 | TMEM16A | Homo sapiens transmembrane protein 16A | TAOS2; ORAOV2; |
| | | | (TMEM16A), mRNA. | FLJ10261 |
| ILMN_14250 | NM_182920.1 | ADAMTS9 | Homo sapiens ADAM metallopeptidase with | KIAA1312 |
| | | | thrombospondin type 1 motif, 9 (ADAMTS9), | |
| | | | mRNA. | |

TABLE 2

| Name | Accession | Symbol | Definition | Synonym |
|---|---|---|---|---|
| ILMN_20088 | NM_001150.1 | ANPEP | Homo sapiens alanyl (membrane) aminopeptidase | CD13; LAP1; PEPN; gp150 |
| | | | (aminopeptidase N, aminopeptidase M, | |
| | | | microsomal | |
| | | | aminopeptidase, CD13, p150) (ANPEP), mRNA. | |
| ILMN_26976 | NM_138375.1 | CABLES1 | Homo sapiens Cdk5 and Abl enzyme substrate 1 | HsT2563; FLJ35924 |
| | | | (CABLES1), mRNA. | |
| ILMN_24376 | NM_022783.1 | DEPDC6 | Homo sapiens DEP domain containing 6 | DEP.6; FLJ12428; |
| | | | (DEPDC6), mRNA. | FLJ13854; DKFZp564B1778 |
| ILMN_17789 | NM_000418.2 | IL4R | Homo sapiens interleukin 4 receptor (IL4R), | CD124; IL4RA |
| | | | transcript variant 1, mRNA. | |
| ILMN_3945 | NM_182734.1 | PLCB1 | Homo sapiens phospholipase C, beta 1 | PLC-I; PI-PLC; PLC-154 |
| | | | (phosphoinositide-specific) (PLCB1), transcript | |
| | | | variant 2, mRNA. | |
| ILMN_27252 | NM_002925.3 | RGS10 | Homo sapiens regulator of G-protein signalling 10 | Impact of Estrogen Receptor |
| | | | (RGS10), transcript variant 2, mRNA. | on Gene Networks |
| | | | | Regulated by Estrogen . . . |
| | | | | Chang 2006 |
| ILMN_7086 | NM_001012661.1 | SLC3A2 | Homo sapiens solute carrier family 3 (activators | 4F2; CD98; MDU1; 4F2HC; |
| | | | of dibasic and neutral amino acid transport), | 4T2HC; NACAE; CD98HC |
| | | | member 2 (SLC3A2), transcript variant 1, | |
| | | | mRNA. | |
| ILMN_25446 | NM_003486.5 | SLC7A5 | Homo sapiens solute carrier family 7 (cationic | E16; CD98; LAT1; 4F2LC; |
| | | | amino acid transporter, y+ system), member 5 | MPE16; hLAT1; D16S469E |
| | | | (SLC7A5), mRNA. | |
| ILMN_28750 | NM_000067.1 | CA2 | Homo sapiens carbonic anhydrase II (CA2), | CA II; CA-II |
| | | | mRNA. | |
| ILMN_4674 | NM_005194.2 | CEBPB | Homo sapiens CCAAT/enhancer binding protein | LAP; CRP2; TCF5; IL6DBP; |
| | | | (C/EBP), beta (CEBPB), mRNA. | NF-IL6; MGC32080; C/EBP- |
| | | | | beta |
| ILMN_6563 | NM_005195.2 | CEBPD | Homo sapiens CCAAT/enhancer binding protein | CELF; CRP3; C/EBP-delta; |
| | | | (C/EBP), delta (CEBPD), mRNA. | NF-IL6-beta |
| ILMN_13615 | NM_004433.3 | ELF3 | Homo sapiens E74-like factor 3 (ets domain | ERT; ESX; EPR-1; ESE-1 |
| | | | transcription factor, epithelial-specific) (ELF3), | |
| | | | mRNA. | |
| ILMN_7287 | NM_031475.1 | ESPN | Homo sapiens espin (ESPN), mRNA. | DFNB36; LP2654; |
| | | | | DKFZP434A196 |
| ILMN_20831 | NM_001450.3 | FHL2 | Homo sapiens four and a half LIM domains 2 | DRAL; AAG11; SLIM3 |
| | | | (FHL2), transcript variant 1, mRNA. | |
| ILMN_25543 | NM_014216.3 | ITPK1 | Homo sapiens inositol 1,3,4-triphosphate 5/6 | ITRPK1 |
| | | | kinase (ITPK1), mRNA. | |
| ILMN_1092 | NM_000228.2 | LAMB3 | Homo sapiens laminin, beta 3 (LAMB3), | LAMNB1 |
| | | | transcript variant 1, mRNA. | |
| ILMN_20333 | NM_020190.2 | OLFML3 | Homo sapiens olfactomedin-like 3 (OLFML3), | OLF44; HNOEL-iso |
| | | | mRNA. | |
| ILMN_21964 | NM_002648.2 | PIM1 | Homo sapiens pim-1 oncogene (PIM1), mRNA. | PIM |
| ILMN_25998 | NM_002285.2 | AFF3 | Homo sapiens AF4/FMR2 family, member 3 | LAF4; MLLT2-like |
| | | | (AFF3), transcript variant 1, mRNA. | |
| ILMN_138313 | NM_001661.2 | ARL4D | Homo sapiens ADP-ribosylation factor-like 4D | ARL6; ARF4L |
| | | | (ARL4D), mRNA. | |
| ILMN_2585 | NM_025080.2 | ASRGL1 | Homo sapiens asparaginase like 1 (ASRGL1), | ALP; ALP1; FLJ22316 |
| | | | mRNA. | |
| ILMN_23075 | NM_003571.2 | BFSP2 | Homo sapiens beaded filament structural protein | CP47; CP49; LIFL-L |
| | | | 2, phakinin (BFSP2), mRNA. | |
| ILMN_934 | NM_016452.1 | CAPN9 | Homo sapiens calpain 9 (CAPN9), transcript | GC36; nCL-4 |
| | | | variant 2, mRNA. | |
| ILMN_11754 | NM_005187.4 | CBFA2T3 | Homo sapiens core-binding factor, runt domain, | ETO2; MTG16; MTGR2; |
| | | | alpha subunit 2; translocated to, 3 (CBFA2T3), | ZMYND4 |
| | | | transcript variant 1, mRNA. | |
| ILMN_1763 | NM_001008708.1 | CHAC2 | Homo sapiens ChaC, cation transport regulator- | — |
| | | | like 2 (E. coli) (CHAC2), mRNA. | |
| ILMN_12477 | NM_003613.2 | CILP | Homo sapiens cartilage intermediate layer protein, | HsT18872 |
| | | | nucleotide pyrophosphohydrolase (CILP), mRNA. | |
| ILMN_12432 | NM_004753.4 | DHRS3 | Homo sapiens dehydrogenase/reductase (SDR | SDR1; Rsdr1; retSDR1 |
| | | | family) member 3 (DHRS3), mRNA. | |
| ILMN_9402 | NM_001003399.1 | DKFZp451A211 | Homo sapiens DKFZp451A211 protein | — |
| | | | (DKFZp451A211), mRNA. | |
| ILMN_5422 | NM_018110.2 | DOK4 | Homo sapiens docking protein 4 (DOK4), | FLJ10488 |
| | | | mRNA. | |
| ILMN_27171 | NM_022073.2 | EGLN3 | Homo sapiens egl nine homolog 3 (C. elegans) | PHD3; HIFPH3; FLJ21620; |
| | | | (EGLN3), mRNA. | MGC125999 |
| ILMN_6138 | NM_001431.1 | EPB41L2 | Homo sapiens erythrocyte membrane protein band | 4.1-G; DKFZp781H1755 |
| | | | 4.1-like 2 (EPB41L2), mRNA. | |
| ILMN_25398 | NM_024841.2 | FLJ14213 | Homo sapiens hypothetical protein FLJ14213 | MGC16218 |
| | | | (FLJ14213), mRNA. | |
| ILMN_138374 | NM_004472.1 | FOXD1 | Homo sapiens forkhead box D1 (FOXD1), | FKHL8; FREAC4 |
| | | | mRNA. | |
| ILMN_26352 | NM_003468.2 | FZD5 | Homo sapiens frizzled homolog 5 (Drosophila) | HFZ5 |
| | | | (FZD5), mRNA. | |
| ILMN_16210 | NM_030792.4 | GDPD5 | Homo sapiens glycerophosphodiester | PP1665 |
| | | | phosphodiesterase domain containing 5 (GDPD5), | |
| | | | mRNA. | |
| ILMN_15240 | NM_019089.3 | HES2 | Homo sapiens hairy and enhancer of split 2 | — |
| | | | (Drosophila) (HES2), mRNA. | |
| ILMN_823 | NM_002236.4 | KCNF1 | Homo sapiens potassium voltage-gated channel, | IK8; kH1; KCNF; KV5.1; |
| | | | subfamily F, member 1 (KCNF1), mRNA. | MGC33316 |
| ILMN_13852 | NM_000890.3 | KCNJ5 | Homo sapiens potassium inwardly-rectifying | CIR; GIRK4; KATP1; KIR3.4 |
| | | | channel, subfamily J, member 5 (KCNJ5), | |
| | | | mRNA. | |
| ILMN_24839 | NM_138370.1 | LOC91461 | Homo sapiens hypothetical protein BC007901 | — |
| | | | (LOC91461), mRNA. | |
| ILMN_29422 | NM_024101.4 | MLPH | Homo sapiens melanophilin (MLPH), mRNA. | In; MELPH; I1Rk3; |
| | | | | MGC2771; SLAC2-A; Slac- |
| | | | | 2a; FLJ12145; I(1)-3Rk; |
| | | | | exophilin-3 |
| ILMN_25657 | NM_004558.2 | NRTN | Homo sapiens neurturin (NRTN), mRNA. | NTN |
| ILMN_29366 | NM_032812.7 | PLXDC2 | Homo sapiens plexin domain containing 2 | TEM7R; FLJ14623 |
| | | | (PLXDC2), mRNA. | |
| ILMN_6412 | NM_153020.1 | RBM24 | Homo sapiens RNA binding motif protein 24 | RNPC6; FLJ30829; |
| | | | (RBM24), mRNA. | FLJ37697; dJ259A10.1 |
| ILMN_9292 | NM_005045.2 | RELN | Homo sapiens reelin (RELN), transcript variant 1, | RL |
| | | | mRNA. | |
| ILMN_25763 | NM_003043.2 | SLC6A6 | Homo sapiens solute carrier family 6 | TAUT; MGC10619 |
| | | | (neurotransmitter transporter, taurine), member 6 | |
| | | | (SLC6A6), mRNA. | |
| ILMN_8034 | NM_022454.2 | SOX17 | Homo sapiens SRY (sex determining region Y)- | FLJ22252 |
| | | | box 17 (SOX17), mRNA. | |
| ILMN_25243 | NM_018043.4 | TMEM16A | Homo sapiens transmembrane protein 16A | TAOS2; ORAOV2; |
| | | | (TMEM16A), mRNA. | FLJ10261 |
| ILMN_14250 | NM_182920.1 | ADAMTS9 | Homo sapiens ADAM metallopeptidase with | KIAA1312 |
| | | | thrombospondin type 1 motif, 9 (ADAMTS9), | |
| | | | mRNA. | |

TABLE 3

| Name | Accession | Symbol | Definition | Synonym |
|---|---|---|---|---|
| ILMN_4188 | NM_003857.2 | GALR2 | Homo sapiens galanin receptor 2 (GALR2), | GALNR2 |
| | | | mRNA. | |
| ILMN_5566 | NM_000422.1 | KRT17 | Homo sapiens keratin 17 (KRT17), mRNA. | PC; K17; PC2; PCHC1 |
| ILMN_11289 | NM_002899.2 | RBP1 | Homo sapiens retinol binding protein 1, cellular | CRBP; RBPC; CRBP1; |
| | | | (RBP1), mRNA. | CRABP-I |
| ILMN_12497 | NM_002970.1 | SAT | Homo sapiens spermidine/spermine N1- | DC21; KFSD; SSAT |
| | | | acetyltransferase (SAT), mRNA. | |
| ILMN_26083 | NM_152321.1 | C12orf46 | Homo sapiens chromosome 12 open reading | FLJ32115 |
| | | | frame 46 (C12orf46), mRNA. | |
| ILMN_29699 | NM_001013625.2 | C1orf192 | Homo sapiens chromosome 1 open reading | — |
| | | | frame 192 | |
| ILMN_7222 | NM_024059.2 | C20orf195 | Homo sapiens chromosome 20 open reading | — |
| | | | frame 195 (C20orf195), mRNA. | |
| ILMN_11032 | NM_022833.2 | C9orf88 | Homo sapiens chromosome 9 open reading | — |
| | | | frame 88 | |
| ILMN_16401 | NM_018272.2 | CASC1 | Homo sapiens cancer susceptibility candidate 1 | — |
| | | | (CASC1), mRNA. | |
| ILMN_11666 | NM_001296.3 | CCBP2 | Homo sapiens chemokine binding protein 2 | D6; hD6; CCR9; CCR10; |
| | | | (CCBP2), mRNA. | CMKBR9 |
| ILMN_138202 | NM_012337.1 | CCDC19 | Homo sapiens coiled-coil domain containing 19 | — |
| | | | (CCDC19), mRNA. | |
| ILMN_137851 | NM_016938.1 | EFEMP2 | Homo sapiens EGF-containing fibulin-like | UPH1; FBLN4 |
| | | | extracellular matrix protein 2 (EFEMP2), | |
| | | | mRNA. | |
| ILMN_18028 | NM_007177.1 | FAM107A | Homo sapiens family with sequence similarity | DRR1; TU3A |
| | | | 107, member A (FAM107A), mRNA. | |
| ILMN_13823 | NM_001454.2 | FOXJ1 | Homo sapiens forkhead box J1 (FOXJ1), | — |
| | | | mRNA. | |
| ILMN_19816 | NM_178232.2 | HAPLN3 | Homo sapiens hyaluronan and proteoglycan | EXLD1; HsT19883 |
| | | | link protein 3 (HAPLN3), mRNA. | |
| ILMN_137320 | NM_004527.2 | MEOX1 | Homo sapiens mesenchyme homeo box 1 | — |
| | | | (MEOX1), transcript variant 1, mRNA. | |
| ILMN_11368 | NM_005928.1 | MFGE8 | Homo sapiens milk fat globule-EGF factor 8 | BA46; EDIL1; OAcGD3S; |
| | | | protein (MFGE8), mRNA. | HsT19888 |
| ILMN_15026 | NM_052880.3 | MGC17330 | Homo sapiens HGFL gene (MGC17330), | — |
| | | | mRNA. | |
| ILMN_26971 | NM_019850.1 | NGEF | Homo sapiens neuronal guanine nucleotide | EPHEXIN |
| | | | exchange factor (NGEF), mRNA. | |
| ILMN_17486 | NM_016335.2 | PRODH | Homo sapiens proline dehydrogenase | PIG6; SCZD4; HSPOX2; |
| | | | (oxidase) 1 (PRODH), nuclear gene encoding | PRODH1; PRODH2; TP53I6 |
| | | | mitochondrial protein, mRNA. | |
| ILMN_13192 | NM_017817.1 | RAB20 | Homo sapiens RAB20, member RAS oncogene | — |
| | | | family (RAB20), mRNA. | |
| ILMN_23427 | NM_004658.1 | RASAL1 | Homo sapiens RAS protein activator like 1 | RASAL |
| | | | (GAP1 like) (RASAL1), mRNA. | |
| ILMN_14916 | NM_148973.1 | TNFRSF25 | Homo sapiens tumor necrosis factor receptor | DR3; TR3; DDR3; LARD; |
| | | | superfamily, member 25 (TNFRSF25), | APO-3; TRAMP; WSL-1; |
| | | | transcript variant 10, mRNA. | WSL-LR; TNFRSF12 |
| ILMN_23986 | NM_015896.2 | ZMYND10 | Homo sapiens zinc finger, MYND-type | — |
| | | | containing 10 (ZMYND10), mRNA. | |

TABLE 4

| Name | Accession | Symbol | Definition | Synonym |
|---|---|---|---|---|
| ILMN_8120 | NM_020037.1 | ABCC3 | Homo sapiens ATP-binding cassette, sub-family C | MLP2; MRP3; ABC31; MOAT- |
| | | | (CFTR/MRP), member 3 (ABCC3), transcript | D; cMOAT2; EST90757 |
| | | | variant MRP3A, mRNA. | |
| ILMN_4380 | NM_000499.2 | CYP1A1 | Homo sapiens cytochrome P450, family 1, | AHH; AHRR; CP11; CYP1; |
| | | | subfamily A, polypeptide 1 (CYP1A1), mRNA. | P1-450; P450-C; P450DX |
| ILMN_23145 | NM_031313.1 | ALPPL2 | Homo sapiens alkaline phosphatase, placental-like | ALPG; GCAP; ALPPL |
| | | | 2 (ALPPL2), mRNA. | |
| ILMN_20319 | NM_014668.2 | GREB1 | Homo sapiens GREB1 protein (GREB1), transcript | KIAA0575 |
| | | | variant a, mRNA. | |
| ILMN_4339 | NM_003489.2 | NRIP1 | Homo sapiens nuclear receptor interacting protein | RIP140 |
| | | | 1 (NRIP1), mRNA. | |
| ILMN_20145 | NM_000926.2 | PGR | Homo sapiens progesterone receptor (PGR), | PR; NR3C3 |
| | | | mRNA. | |
| ILMN_30018 | NM_003236.1 | TGFA | Homo sapiens transforming growth factor, alpha | TFGA |
| | | | (TGFA), mRNA. | |
| ILMN_18256 | NM_005226.2 | EDG3 | Homo sapiens endothelial differentiation, | LPB3; S1P3; EDG-3; S1PR3; |
| | | | sphingolipid G-protein-coupled receptor, 3 | FLJ37523; MGC71696 |
| | | | (EDG3), mRNA. | |
| ILMN_21428 | NM_015714.2 | G0S2 | Homo sapiens G0/G1switch 2 (G0S2), mRNA. | RP1-28O10.2 |
| ILMN_15303 | NM_003774.3 | GALNT4 | Homo sapiens UDP-N-acetyl-alpha-D- | GalNAcT4; GALNAC-T4 |
| | | | galactosamine:polypeptide N- | |
| | | | acetylgalactosaminyltransferase 4 (GalNAc-T4) | |
| | | | (GALNT4), mRNA. | |
| ILMN_27341 | NM_014279.2 | OLFM1 | Homo sapiens olfactomedin 1 (OLFM1), transcript | AMY; NOE1; OlfA; NOELIN; |
| | | | variant 1, mRNA. | NOELIN1; NOELIN1_V1; |
| | | | | NOELIN1_V2; NOELIN1_V4 |
| ILMN_1435 | NM_006334.2 | OLFM1 | Homo sapiens olfactomedin 1 (OLFM1), transcript | AMY; NOE1; OlfA; NOELIN; |
| | | | variant 2, mRNA. | NOELIN1; NOELIN1_V1; |
| | | | | NOELIN1_V2; NOELIN1_V4 |
| ILMN_21535 | NM_005067.5 | SIAH2 | Homo sapiens seven in absentia homolog 2 | hSiah2 |
| | | | (Drosophila) (SIAH2), mRNA. | |
| ILMN_4882 | NM_003246.2 | THBS1 | Homo sapiens thrombospondin 1 (THBS1), | TSP; THBS; TSP1 |
| | | | mRNA. | |
| ILMN_18279 | NM_004185.2 | WNT2B | Homo sapiens wingless-type MMTV integration | WNT13; XWNT2 |
| | | | site family, member 2B (WNT2B), transcript | |
| | | | variant WNT-2B1, mRNA. | |

TABLE 5

| Name | Accession | Symbol | Definition | Synonym |
|---|---|---|---|---|
| ILMN_23145 | NM_031313.1 | ALPPL2 | Homo sapiens alkaline phosphatase, placental-like | ALPG; GCAP; ALPPL |
| | | | 2 (ALPPL2), mRNA. | |
| ILMN_20319 | NM_014668.2 | GREB1 | Homo sapiens GREB1 protein (GREB1), transcript | KIAA0575 |
| | | | variant a, mRNA. | |
| ILMN_4339 | NM_003489.2 | NRIP1 | Homo sapiens nuclear receptor interacting protein | RIP140 |
| | | | 1 (NRIP1), mRNA. | |
| ILMN_20145 | NM_000926.2 | PGR | Homo sapiens progesterone receptor (PGR), | PR; NR3C3 |
| | | | mRNA. | |
| ILMN_30018 | NM_003236.1 | TGFA | Homo sapiens transforming growth factor, alpha | TFGA |
| | | | (TGFA), mRNA. | |
| ILMN_18256 | NM_005226.2 | EDG3 | Homo sapiens endothelial differentiation, | LPB3; S1P3; EDG-3; S1PR3; |
| | | | sphingolipid G-protein-coupled receptor, 3 | FLJ37523; MGC71696 |
| | | | (EDG3), mRNA. | |
| ILMN_21428 | NM_015714.2 | G0S2 | Homo sapiens G0/G1switch 2 (G0S2), mRNA. | RP1-28O10.2 |
| ILMN_15303 | NM_003774.3 | GALNT4 | Homo sapiens UDP-N-acetyl-alpha-D- | GalNAcT4; GALNAC-T4 |
| | | | galactosamine:polypeptide N- | |
| | | | acetylgalactosaminyltransferase 4 (GalNAc-T4) | |
| | | | (GALNT4), mRNA. | |
| ILMN_27341 | NM_014279.2 | OLFM1 | Homo sapiens olfactomedin 1 (OLFM1), transcript | AMY; NOE1; OlfA; NOELIN; |
| | | | variant 1, mRNA. | NOELIN1; NOELIN1_V1; |
| | | | | NOELIN1_V2; NOELIN1_V4 |
| ILMN_1435 | NM_006334.2 | OLFM1 | Homo sapiens olfactomedin 1 (OLFM1), transcript | AMY; NOE1; OlfA; NOELIN; |
| | | | variant 2, mRNA. | NOELIN1; NOELIN1_V1; |
| | | | | NOELIN1_V2; NOELIN1_V4 |
| ILMN_21535 | NM_005067.5 | SIAH2 | Homo sapiens seven in absentia homolog 2 | hSiah2 |
| | | | (Drosophila) (SIAH2), mRNA. | |
| ILMN_4882 | NM_003246.2 | THBS1 | Homo sapiens thrombospondin 1 (THBS1), | TSP; THBS; TSP1 |
| | | | mRNA. | |
| ILMN_18279 | NM_004185.2 | WNT2B | Homo sapiens wingless-type MMTV integration | WNT13; XWNT2 |
| | | | site family, member 2B (WNT2B), transcript | |
| | | | variant WNT-2B1, mRNA. | |

TABLE 6

| Name | Accession | Symbol | Definition | Synonym |
|---|---|---|---|---|
| ILMN_8120 | NM_020037.1 | ABCC3 | Homo sapiens ATP-binding cassette, | MLP2; MRP3; ABC31; |
| | | | sub-family C (CFTR/MRP), | MOAT-D; cMOAT2; |
| | | | member 3 (ABCC3), transcript variant | EST90757 |
| | | | MRP3A, mRNA. | |
| ILMN_4380 | NM_000499.2 | CYP1A1 | Homo sapiens cytochrome P450, | AHH; AHRR; CP11; |
| | | | family 1, subfamily A, polypeptide 1 | CYP1; P1-450; P450- |
| | | | (CYP1A1), mRNA. | C; P450DX |

TABLE 7

| Name | Accession | Symbol | Definition | Synonym |
|---|---|---|---|---|
| ILMN_26083 | NM_152321.1 | C12orf46 | Homo sapiens chromosome 12 open reading | FLJ32115 |
| | | | frame 46 (C12orf46), mRNA. | |
| ILMN_29699 | NM_001013625.2 | C1orf192 | Homo sapiens chromosome 1 open reading | — |
| | | | frame 192 | |
| ILMN_7222 | NM_024059.2 | C20orf195 | Homo sapiens chromosome 20 open reading | — |
| | | | frame 195 (C20orf195), mRNA. | |
| ILMN_11032 | NM_022833.2 | C9orf88 | Homo sapiens chromosome 9 open reading | — |
| | | | frame 88 | |
| ILMN_16401 | NM_018272.2 | CASC1 | Homo sapiens cancer susceptibility | — |
| | | | candidate 1 (CASC1), mRNA. | |
| ILMN_11666 | NM_001296.3 | CCBP2 | Homo sapiens chemokine binding protein 2 | D6; hD6; CCR9; |
| | | | (CCBP2), mRNA. | CCR10; CMKBR9 |
| ILMN_138202 | NM_012337.1 | CCDC19 | Homo sapiens coiled-coil domain containing | — |
| | | | 19 (CCDC19), mRNA. | |
| ILMN_137851 | NM_016938.1 | EFEMP2 | Homo sapiens EGF-containing fibulin-like | UPH1; FBLN4 |
| | | | extracellular matrix protein 2 (EFEMP2), | |
| | | | mRNA. | |
| ILMN_18028 | NM_007177.1 | FAM107A | Homo sapiens family with sequence similarity | DRR1; TU3A |
| | | | 107, member A (FAM107A), mRNA. | |
| ILMN_13823 | NM_001454.2 | FOXJ1 | Homo sapiens forkhead box J1 (FOXJ1), | — |
| | | | mRNA. | |
| ILMN_19816 | NM_178232.2 | HAPLN3 | Homo sapiens hyaluronan and proteoglycan | EXLD1; HsT19883 |
| | | | link protein 3 (HAPLN3), mRNA. | |
| ILMN_137320 | NM_004527.2 | MEOX1 | Homo sapiens mesenchyme homeo box 1 | — |
| | | | (MEOX1), transcript variant 1, mRNA. | |
| ILMN_11368 | NM_005928.1 | MFGE8 | Homo sapiens milk fat globule-EGF factor 8 | BA46; EDIL1; |
| | | | protein (MFGE8), mRNA. | OAcGD3S; |
| | | | | HsT19888 |
| ILMN_15026 | NM_052880.3 | MGC17330 | Homo sapiens HGFL gene (MGC17330), | — |
| | | | mRNA. | |
| ILMN_26971 | NM_019850.1 | NGEF | Homo sapiens neuronal guanine nucleotide | EPHEXIN |
| | | | exchange factor (NGEF), mRNA. | |
| ILMN_17486 | NM_016335.2 | PRODH | Homo sapiens proline dehydrogenase | PIG6; SCZD4; |
| | | | (oxidase) 1 (PRODH), nuclear gene encoding | HSPOX2; |
| | | | mitochondrial protein, mRNA. | PRODH1; |
| | | | | PRODH2; TP53I6 |
| ILMN_13192 | NM_017817.1 | RAB20 | Homo sapiens RAB20, member RAS | — |
| | | | oncogene family (RAB20), mRNA. | |
| ILMN_23427 | NM_004658.1 | RASAL1 | Homo sapiens RAS protein activator like 1 | RASAL |
| | | | (GAP1 like) (RASAL1), mRNA. | |
| ILMN_23986 | NM_015896.2 | ZMYND10 | Homo sapiens zinc finger, MYND-type | — |
| | | | containing 10 (ZMYND10), mRNA. | |
| ILMN_25998 | NM_002285.2 | AFF3 | Homo sapiens AF4/FMR2 family, member 3 | LAF4; MLLT2-like |
| | | | (AFF3), transcript variant 1, mRNA. | |
| ILMN_138313 | NM_001661.2 | ARL4D | Homo sapiens ADP-ribosylation factor-like | ARL6; ARF4L |
| | | | 4D (ARL4D), mRNA. | |
| ILMN_2585 | NM_025080.2 | ASRGL1 | Homo sapiens asparaginase like 1 | ALP; ALP1; |
| | | | (ASRGL1), mRNA. | FLJ22316 |
| ILMN_23075 | NM_003571.2 | BFSP2 | Homo sapiens beaded filament structural | CP47; CP49; LIFL-L |
| | | | protein 2, phakinin (BFSP2), mRNA. | |
| ILMN_934 | NM_016452.1 | CAPN9 | Homo sapiens calpain 9 (CAPN9), transcript | GC36; nCL-4 |
| | | | variant 2, mRNA. | |
| ILMN_11754 | NM_005187.4 | CBFA2T3 | Homo sapiens core-binding factor, runt | ETO2; MTG16; |
| | | | domain, alpha subunit 2; translocated to, 3 | MTGR2; ZMYND4 |
| | | | (CBFA2T3), transcript variant 1, mRNA. | |
| ILMN_1763 | NM_001008708.1 | CHAC2 | Homo sapiens ChaC, cation transport | — |
| | | | regulator-like 2 (E. coli) (CHAC2), mRNA. | |
| ILMN_12477 | NM_003613.2 | CILP | Homo sapiens cartilage intermediate layer | HsT18872 |
| | | | protein, nucleotide pyrophosphohydrolase | |
| | | | (CILP), mRNA. | |
| ILMN_12432 | NM_004753.4 | DHRS3 | Homo sapiens dehydrogenase/reductase | SDR1; Rsdr1; |
| | | | (SDR family) member 3 (DHRS3), mRNA. | retSDR1 |
| ILMN_9402 | NM_001003399.1 | DKFZp451A211 | Homo sapiens DKFZp451A211 protein | — |
| | | | (DKFZp451A211), mRNA. | |
| ILMN_5422 | NM_018110.2 | DOK4 | Homo sapiens docking protein 4 (DOK4), | FLJ10488 |
| | | | mRNA. | |
| ILMN_27171 | NM_022073.2 | EGLN3 | Homo sapiens egl nine homolog 3 (C. elegans) | PHD3; HIFPH3; |
| | | | (EGLN3), mRNA. | FLJ21620; |
| | | | | MGC125999 |
| ILMN_6138 | NM_001431.1 | EPB41L2 | Homo sapiens erythrocyte membrane protein | 4.1-G; |
| | | | band 4.1-like 2 (EPB41L2), mRNA. | DKFZp781H1755 |
| ILMN_25398 | NM_024841.2 | FLJ14213 | Homo sapiens hypothetical protein FLJ14213 | MGC16218 |
| | | | (FLJ14213), mRNA. | |
| ILMN_138374 | NM_004472.1 | FOXD1 | Homo sapiens forkhead box D1 (FOXD1), | FKHL8; FREAC4 |
| | | | mRNA. | |
| ILMN_26352 | NM_003468.2 | FZD5 | Homo sapiens frizzled homolog 5 | HFZ5 |
| | | | (Drosophila) (FZD5), mRNA. | |
| ILMN_16210 | NM_030792.4 | GDPD5 | Homo sapiens glycerophosphodiester | PP1665 |
| | | | phosphodiesterase domain containing 5 | |
| | | | (GDPD5), mRNA. | |
| ILMN_15240 | NM_019089.3 | HES2 | Homo sapiens hairy and enhancer of split 2 | — |
| | | | (Drosophila) (HES2), mRNA. | |
| ILMN_823 | NM_002236.4 | KCNF1 | Homo sapiens potassium voltage-gated | IK8; kH1; KCNF; |
| | | | channel, subfamily F, member 1 (KCNF1), | KV5.1; MGC33316 |
| | | | mRNA. | |
| ILMN_13852 | NM_000890.3 | KCNJ5 | Homo sapiens potassium inwardly-rectifying | CIR; GIRK4; |
| | | | channel, subfamily J, member 5 (KCNJ5), | KATP1; KIR3.4 |
| | | | mRNA. | |
| ILMN_24839 | NM_138370.1 | LOC91461 | Homo sapiens hypothetical protein | — |
| | | | BC007901 (LOC91461), mRNA. | |
| ILMN_29422 | NM_024101.4 | MLPH | Homo sapiens melanophilin (MLPH), mRNA. | In; MELPH; I1Rk3; |
| | | | | MGC2771; SLAC2- |
| | | | | A; Slac-2a; |
| | | | | FLJ12145; I(1)-3Rk; |
| | | | | exophilin-3 |
| ILMN_25657 | NM_004558.2 | NRTN | Homo sapiens neurturin (NRTN), mRNA. | NTN |
| ILMN_29366 | NM_032812.7 | PLXDC2 | Homo sapiens plexin domain containing 2 | TEM7R; FLJ14623 |
| | | | (PLXDC2), mRNA. | |
| ILMN_6412 | NM_153020.1 | RBM24 | Homo sapiens RNA binding motif protein 24 | RNPC6; FLJ30829; |
| | | | (RBM24), mRNA. | FLJ37697; |
| | | | | dJ259A10.1 |
| ILMN_9292 | NM_005045.2 | RELN | Homo sapiens reelin (RELN), transcript | RL |
| | | | variant 1, mRNA. | |
| ILMN_25763 | NM_003043.2 | SLC6A6 | Homo sapiens solute carrier family 6 | TAUT; MGC10619 |
| | | | (neurotransmitter transporter, taurine), | |
| | | | member 6 (SLC6A6), mRNA. | |
| ILMN_8034 | NM_022454.2 | SOX17 | Homo sapiens SRY (sex determining region | FLJ22252 |
| | | | Y)-box 17 (SOX17), mRNA. | |
| ILMN_25243 | NM_018043.4 | TMEM16A | Homo sapiens transmembrane protein 16A | TAOS2; ORAOV2; |
| | | | (TMEM16A), mRNA. | FLJ10261 |
| ILMN_14250 | NM_182920.1 | ADAMTS9 | Homo sapiens ADAM metallopeptidase with | KIAA1312 |
| | | | thrombospondin type 1 motif, 9 (ADAMTS9), | |
| | | | mRNA. | |

TABLE 8

| Name | Accession | Symbol | Definition | Synonym |
|---|---|---|---|---|
| ILMN_25998 | NM_002285.2 | AFF3 | Homo sapiens AF4/FMR2 family, member 3 | LAF4; MLLT2-like |
| | | | (AFF3), transcript variant 1, mRNA. | |
| ILMN_138313 | NM_001661.2 | ARL4D | Homo sapiens ADP-ribosylation factor-like | ARL6; ARF4L |
| | | | 4D (ARL4D), mRNA. | |
| ILMN_2585 | NM_025080.2 | ASRGL1 | Homo sapiens asparaginase like 1 | ALP; ALP1; |
| | | | (ASRGL1), mRNA. | FLJ22316 |
| ILMN_23075 | NM_003571.2 | BFSP2 | Homo sapiens beaded filament structural | CP47; CP49; LIFL-L |
| | | | protein 2, phakinin (BFSP2), mRNA. | |
| ILMN_934 | NM_016452.1 | CAPN9 | Homo sapiens calpain 9 (CAPN9), transcript | GC36; nCL-4 |
| | | | variant 2, mRNA. | |
| ILMN_11754 | NM_005187.4 | CBFA2T3 | Homo sapiens core-binding factor, runt | ETO2; MTG16; |
| | | | domain, alpha subunit 2; translocated to, 3 | MTGR2; ZMYND4 |
| | | | (CBFA2T3), transcript variant 1, mRNA. | |
| ILMN_1763 | NM_001008708.1 | CHAC2 | Homo sapiens ChaC, cation transport | — |
| | | | regulator-like 2 (E. coli) (CHAC2), mRNA. | |
| ILMN_12477 | NM_003613.2 | CILP | Homo sapiens cartilage intermediate layer | HsT18872 |
| | | | protein, nucleotide pyrophosphohydrolase | |
| | | | (CILP), mRNA. | |
| ILMN_12432 | NM_004753.4 | DHRS3 | Homo sapiens dehydrogenase/reductase | SDR1; Rsdr1; |
| | | | (SDR family) member 3 (DHRS3), mRNA. | retSDR1 |
| ILMN_9402 | NM_001003399.1 | DKFZp451A211 | Homo sapiens DKFZp451A211 protein | — |
| | | | (DKFZp451A211), mRNA. | |
| ILMN_5422 | NM_018110.2 | DOK4 | Homo sapiens docking protein 4 (DOK4), | FLJ10488 |
| | | | mRNA. | |
| ILMN_27171 | NM_022073.2 | EGLN3 | Homo sapiens egl nine homolog 3 (C. elegans) | PHD3; HIFPH3; |
| | | | (EGLN3), mRNA. | FLJ21620; |
| | | | | MGC125999 |
| ILMN_6138 | NM_001431.1 | EPB41L2 | Homo sapiens erythrocyte membrane protein | 4.1-G; |
| | | | band 4.1-like 2 (EPB41L2), mRNA. | DKFZp781H1755 |
| ILMN_25398 | NM_024841.2 | FLJ14213 | Homo sapiens hypothetical protein FLJ14213 | MGC16218 |
| | | | (FLJ14213), mRNA. | |
| ILMN_138374 | NM_004472.1 | FOXD1 | Homo sapiens forkhead box D1 (FOXD1), | FKHL8; FREAC4 |
| | | | mRNA. | |
| ILMN_26352 | NM_003468.2 | FZD5 | Homo sapiens frizzled homolog 5 | HFZ5 |
| | | | (Drosophila) (FZD5), mRNA. | |
| ILMN_16210 | NM_030792.4 | GDPD5 | Homo sapiens glycerophosphodiester | PP1665 |
| | | | phosphodiesterase domain containing 5 | |
| | | | (GDPD5), mRNA. | |
| ILMN_15240 | NM_019089.3 | HES2 | Homo sapiens hairy and enhancer of split 2 | — |
| | | | (Drosophila) (HES2), mRNA. | |
| ILMN_823 | NM_002236.4 | KCNF1 | Homo sapiens potassium voltage-gated | IK8; kH1; KCNF; |
| | | | channel, subfamily F, member 1 (KCNF1), | KV5.1; MGC33316 |
| | | | mRNA. | |
| ILMN_13852 | NM_000890.3 | KCNJ5 | Homo sapiens potassium inwardly-rectifying | CIR; GIRK4; |
| | | | channel, subfamily J, member 5 (KCNJ5), | KATP1; KIR3.4 |
| | | | mRNA. | |
| ILMN_24839 | NM_138370.1 | LOC91461 | Homo sapiens hypothetical protein | — |
| | | | BC007901 (LOC91461), mRNA. | |
| ILMN_29422 | NM_024101.4 | MLPH | Homo sapiens melanophilin (MLPH), mRNA. | In; MELPH; I1Rk3; |
| | | | | MGC2771; SLAC2- |
| | | | | A; Slac-2a; |
| | | | | FLJ12145; I(1)-3Rk; |
| | | | | exophilin-3 |
| ILMN_25657 | NM_004558.2 | NRTN | Homo sapiens neurturin (NRTN), mRNA. | NTN |
| ILMN_29366 | NM_032812.7 | PLXDC2 | Homo sapiens plexin domain containing 2 | TEM7R; FLJ14623 |
| | | | (PLXDC2), mRNA. | |
| ILMN_6412 | NM_153020.1 | RBM24 | Homo sapiens RNA binding motif protein 24 | RNPC6; FLJ30829; |
| | | | (RBM24), mRNA. | FLJ37697; |
| | | | | dJ259A10.1 |
| ILMN_9292 | NM_005045.2 | RELN | Homo sapiens reelin (RELN), transcript | RL |
| | | | variant 1, mRNA. | |
| ILMN_25763 | NM_003043.2 | SLC6A6 | Homo sapiens solute carrier family 6 | TAUT; MGC10619 |
| | | | (neurotransmitter transporter, taurine), | |
| | | | member 6 (SLC6A6), mRNA. | |
| ILMN_8034 | NM_022454.2 | SOX17 | Homo sapiens SRY (sex determining region | FLJ22252 |
| | | | Y)-box 17 (SOX17), mRNA. | |
| ILMN_25243 | NM_018043.4 | TMEM16A | Homo sapiens transmembrane protein 16A | TAOS2; ORAOV2; |
| | | | (TMEM16A), mRNA. | FLJ10261 |
| ILMN_14250 | NM_182920.1 | ADAMTS9 | Homo sapiens ADAM metallopeptidase with | KIAA1312 |
| | | | thrombospondin type 1 motif, 9 (ADAMTS9), | |
| | | | mRNA. | |

TABLE 9

| Name | Accession | Symbol | Definition | Synonym |
|---|---|---|---|---|
| ILMN_26083 | NM_152321.1 | C12orf46 | Homo sapiens chromosome 12 open reading | FLJ32115 |
| | | | frame 46 (C12orf46), mRNA. | |
| ILMN_29699 | NM_001013625.2 | C1orf192 | Homo sapiens chromosome 1 open reading | — |
| | | | frame 192 | |
| ILMN_7222 | NM_024059.2 | C20orf195 | Homo sapiens chromosome 20 open reading | — |
| | | | frame 195 (C20orf195), mRNA. | |
| ILMN_11032 | NM_022833.2 | C9orf88 | Homo sapiens chromosome 9 open reading | — |
| | | | frame 88 | |
| ILMN_16401 | NM_018272.2 | CASC1 | Homo sapiens cancer susceptibility | — |
| | | | candidate 1 (CASC1), mRNA. | |
| ILMN_11666 | NM_001296.3 | CCBP2 | Homo sapiens chemokine binding protein 2 | D6; hD6; CCR9; |
| | | | (CCBP2), mRNA. | CCR10; CMKBR9 |
| ILMN_138202 | NM_012337.1 | CCDC19 | Homo sapiens coiled-coil domain containing | — |
| | | | 19 (CCDC19), mRNA. | |
| ILMN_137851 | NM_016938.1 | EFEMP2 | Homo sapiens EGF-containing fibulin-like | UPH1; FBLN4 |
| | | | extracellular matrix protein 2 (EFEMP2), | |
| | | | mRNA. | |
| ILMN_18028 | NM_007177.1 | FAM107A | Homo sapiens family with sequence similarity | DRR1; TU3A |
| | | | 107, member A (FAM107A), mRNA. | |
| ILMN_13823 | NM_001454.2 | FOXJ1 | Homo sapiens forkhead box J1 (FOXJ1), | — |
| | | | mRNA. | |
| ILMN_19816 | NM_178232.2 | HAPLN3 | Homo sapiens hyaluronan and proteoglycan | EXLD1; HsT19883 |
| | | | link protein 3 (HAPLN3), mRNA. | |
| ILMN_137320 | NM_004527.2 | MEOX1 | Homo sapiens mesenchyme homeo box 1 | — |
| | | | (MEOX1), transcript variant 1, mRNA. | |
| ILMN_11368 | NM_005928.1 | MFGE8 | Homo sapiens milk fat globule-EGF factor 8 | BA46; EDIL1; |
| | | | protein (MFGE8), mRNA. | OAcGD3S; |
| | | | | HsT19888 |
| ILMN_15026 | NM_052880.3 | MGC17330 | Homo sapiens HGFL gene (MGC17330), | — |
| | | | mRNA. | |
| ILMN_26971 | NM_019850.1 | NGEF | Homo sapiens neuronal guanine nucleotide | EPHEXIN |
| | | | exchange factor (NGEF), mRNA. | |
| ILMN_17486 | NM_016335.2 | PRODH | Homo sapiens proline dehydrogenase | PIG6; SCZD4; |
| | | | (oxidase) 1 (PRODH), nuclear gene encoding | HSPOX2; |
| | | | mitochondrial protein, mRNA. | PRODH1; |
| | | | | PRODH2; TP53I6 |
| ILMN_13192 | NM_017817.1 | RAB20 | Homo sapiens RAB20, member RAS | — |
| | | | oncogene family (RAB20), mRNA. | |
| ILMN_23427 | NM_004658.1 | RASAL1 | Homo sapiens RAS protein activator like 1 | RASAL |
| | | | (GAP1 like) (RASAL1), mRNA. | |
| ILMN_23986 | NM_015896.2 | ZMYND10 | Homo sapiens zinc finger, MYND-type | — |
| | | | containing 10 (ZMYND10), mRNA. | |

TABLE 10

| Name | Accession | Symbol | Definition | Synonym |
|---|---|---|---|---|
| ILMN_23145 | NM_031313.1 | ALPPL2 | Homo sapiens alkaline | ALPG; GCAP; |
| | | | phosphatase, placental-like 2 | ALPPL |
| | | | (ALPPL2), mRNA. | |
| ILMN_20319 | NM_014668.2 | GREB1 | Homo sapiens GREB1 protein | KIAA0575 |
| | | | (GREB1), transcript variant a, | |
| | | | mRNA. | |
| ILMN_30018 | NM_003236.1 | TGFA | Homo sapiens transforming | TFGA |
| | | | growth factor, alpha (TGFA), | |
| | | | mRNA. | |
| ILMN_18256 | NM_005226.2 | EDG3 | Homo sapiens endothelial | LPB3; S1P3; EDG- |
| | | | differentiation, sphingolipid G- | 3; S1PR3; |
| | | | protein-coupled receptor, 3 | FLJ37523; |
| | | | (EDG3), mRNA. | MGC71696 |
| ILMN_21428 | NM_015714.2 | G0S2 | Homo sapiens G0/G1switch 2 | RP1-28O10.2 |
| | | | (G0S2), mRNA. | |
| ILMN_15303 | NM_003774.3 | GALNT4 | Homo sapiens UDP-N-acetyl- | GalNAcT4; |
| | | | alpha-D- | GALNAC-T4 |
| | | | galactosamine:polypeptide N- | |
| | | | acetylgalactosaminyltransferase 4 | |
| | | | (GalNAc-T4) (GALNT4), mRNA. | |
| ILMN_27341 | NM_014279.2 | OLFM1 | Homo sapiens olfactomedin 1 | AMY; NOE1; OlfA; |
| | | | (OLFM1), transcript variant 1, | NOELIN; NOELIN1; |
| | | | mRNA. | NOELIN1_V1; |
| | | | | NOELIN1_V2; |
| | | | | NOELIN1_V4 |
| ILMN_1435 | NM_006334.2 | OLFM1 | Homo sapiens olfactomedin 1 | AMY; NOE1; OlfA; |
| | | | (OLFM1), transcript variant 2, | NOELIN; NOELIN1; |
| | | | mRNA. | NOELIN1_V1; |
| | | | | NOELIN1_V2; |
| | | | | NOELIN1_V4 |
| ILMN_21535 | NM_005067.5 | SIAH2 | Homo sapiens seven in absentia | hSiah2 |
| | | | homolog 2 (Drosophila) (SIAH2), | |
| | | | mRNA. | |
| ILMN_4882 | NM_003246.2 | THBS1 | Homo sapiens thrombospondin 1 | TSP; THBS; TSP1 |
| | | | (THBS1), mRNA. | |
| ILMN_18279 | NM_004185.2 | WNT2B | Homo sapiens wingless-type | WNT13; XWNT2 |
| | | | MMTV integration site family, | |
| | | | member 2B (WNT2B), transcript | |
| | | | variant WNT-2B1, mRNA. | |
| ILMN_20088 | NM_001150.1 | ANPEP | Homo sapiens alanyl (membrane) | CD13; LAP1; |
| | | | aminopeptidase (aminopeptidase | PEPN; gp150 |
| | | | N, aminopeptidase M, microsomal | |
| | | | aminopeptidase, CD13, p150) | |
| | | | (ANPEP), mRNA. | |
| ILMN_26976 | NM_138375.1 | CABLES1 | Homo sapiens Cdk5 and Abl | HsT2563; FLJ35924 |
| | | | enzyme substrate 1 (CABLES1), | |
| | | | mRNA. | |
| ILMN_24376 | NM_022783.1 | DEPDC6 | Homo sapiens DEP domain | DEP.6; FLJ12428; |
| | | | containing 6 (DEPDC6), mRNA. | FLJ13854; |
| | | | | DKFZp564B1778 |
| ILMN_17789 | NM_000418.2 | IL4R | Homo sapiens interleukin 4 | CD124; IL4RA |
| | | | receptor (IL4R), transcript variant | |
| | | | 1, mRNA. | |
| ILMN_3945 | NM_182734.1 | PLCB1 | Homo sapiens phospholipase C, | PLC-I; PI-PLC; |
| | | | beta 1 (phosphoinositide-specific) | PLC-154 |
| | | | (PLCB1), transcript variant 2, | |
| | | | mRNA. | |
| ILMN_27252 | NM_002925.3 | RGS10 | Homo sapiens regulator of G- | Impact of Estrogen |
| | | | protein signalling 10 (RGS10), | Receptor on Gene |
| | | | transcript variant 2, mRNA. | Networks Regulated |
| | | | | by Estrogen . . . |
| | | | | Chang 2006 |
| ILMN_7086 | NM_001012661.1 | SLC3A2 | Homo sapiens solute carrier family | 4F2; CD98; MDU1; |
| | | | 3 (activators of dibasic and neutral | 4F2HC; 4T2HC; |
| | | | amino acid transport), member 2 | NACAE; CD98HC |
| | | | (SLC3A2), transcript variant 1, | |
| | | | mRNA. | |
| ILMN_25446 | NM_003486.5 | SLC7A5 | Homo sapiens solute carrier family | E16; CD98; LAT1; |
| | | | 7 (cationic amino acid transporter, | 4F2LC; MPE16; |
| | | | y+ system), member 5 (SLC7A5), | hLAT1; D16S469E |
| | | | mRNA. | |
| ILMN_28750 | NM_000067.1 | CA2 | Homo sapiens carbonic anhydrase | CA II; CA-II |
| | | | II (CA2), mRNA. | |
| ILMN_4674 | NM_005194.2 | CEBPB | Homo sapiens CCAAT/enhancer | LAP; CRP2; TCF5; |
| | | | binding protein (C/EBP), beta | IL6DBP; NF-IL6; |
| | | | (CEBPB), mRNA. | MGC32080; C/EBP- |
| | | | | beta |
| ILMN_6563 | NM_005195.2 | CEBPD | Homo sapiens CCAAT/enhancer | CELF; CRP3; |
| | | | binding protein (C/EBP), delta | C/EBP-delta; NF- |
| | | | (CEBPD), mRNA. | IL6-beta |
| ILMN_13615 | NM_004433.3 | ELF3 | Homo sapiens E74-like factor 3 | ERT; ESX; EPR-1; |
| | | | (ets domain transcription factor, | ESE-1 |
| | | | epithelial-specific) (ELF3), mRNA. | |
| ILMN_7287 | NM_031475.1 | ESPN | Homo sapiens espin (ESPN), | DFNB36; LP2654; |
| | | | mRNA. | DKFZP434A196 |
| ILMN_20831 | NM_001450.3 | FHL2 | Homo sapiens four and a half LIM | DRAL; AAG11; |
| | | | domains 2 (FHL2), transcript | SLIM3 |
| | | | variant 1, mRNA. | |
| ILMN_25543 | NM_014216.3 | ITPK1 | Homo sapiens inositol 1,3,4- | ITRPK1 |
| | | | triphosphate 5/6 kinase (ITPK1), | |
| | | | mRNA. | |
| ILMN_1092 | NM_000228.2 | LAMB3 | Homo sapiens laminin, beta 3 | LAMNB1 |
| | | | (LAMB3), transcript variant 1, | |
| | | | mRNA. | |
| ILMN_20333 | NM_020190.2 | OLFML3 | Homo sapiens olfactomedin-like 3 | OLF44; HNOEL-iso |
| | | | (OLFML3), mRNA. | |
| ILMN_21964 | NM_002648.2 | PIM1 | Homo sapiens pim-1 oncogene | PIM |
| | | | (PIM1), mRNA. | |
| ILMN_4188 | NM_003857.2 | GALR2 | Homo sapiens galanin receptor 2 | GALNR2 |
| | | | (GALR2), mRNA. | |
| ILMN_5566 | NM_000422.1 | KRT17 | Homo sapiens keratin 17 (KRT17), | PC; K17; PC2; |
| | | | mRNA. | PCHC1 |
| ILMN_11289 | NM_002899.2 | RBP1 | Homo sapiens retinol binding | CRBP; RBPC; |
| | | | protein 1, cellular (RBP1), mRNA. | CRBP1; CRABP-I |
| ILMN_12497 | NM_002970.1 | SAT | Homo sapiens | DC21; KFSD; SSAT |
| | | | spermidine/spermine N1- | |
| | | | acetyltransferase (SAT), mRNA. | |
| ILMN_14916 | NM_148973.1 | TNFRSF25 | Homo sapiens tumor necrosis | DR3; TR3; DDR3; |
| | | | factor receptor superfamily, | LARD; APO-3; |
| | | | member 25 (TNFRSF25), | TRAMP; WSL-1; |
| | | | transcript variant 10, mRNA. | WSL-LR; |
| | | | | TNFRSF12 |

TABLE 11

| Name | Accession | Symbol | Definition | Synonym |
|---|---|---|---|---|
| ILMN_23145 | NM_031313.1 | ALPPL2 | Homo sapiens alkaline | ALPG; GCAP; |
| | | | phosphatase, placental-like 2 | ALPPL |
| | | | (ALPPL2), mRNA. | |
| ILMN_20319 | NM_014668.2 | GREB1 | Homo sapiens GREB1 protein | KIAA0575 |
| | | | (GREB1), transcript variant a, | |
| | | | mRNA. | |
| ILMN_30018 | NM_003236.1 | TGFA | Homo sapiens transforming | TFGA |
| | | | growth factor, alpha (TGFA), | |
| | | | mRNA. | |
| ILMN_18256 | NM_005226.2 | EDG3 | Homo sapiens endothelial | LPB3; S1P3; EDG- |
| | | | differentiation, sphingolipid G- | 3; S1PR3; |
| | | | protein-coupled receptor, 3 | FLJ37523; |
| | | | (EDG3), mRNA. | MGC71696 |
| ILMN_21428 | NM_015714.2 | G0S2 | Homo sapiens G0/G1switch 2 | RP1-28O10.2 |
| | | | (G0S2), mRNA. | |
| ILMN_15303 | NM_003774.3 | GALNT4 | Homo sapiens UDP-N-acetyl- | GalNAcT4; |
| | | | alpha-D- | GALNAC-T4 |
| | | | galactosamine:polypeptide N- | |
| | | | acetylgalactosaminyltransferase 4 | |
| | | | (GalNAc-T4) (GALNT4), mRNA. | |
| ILMN_27341 | NM_014279.2 | OLFM1 | Homo sapiens olfactomedin 1 | AMY; NOE1; OlfA; |
| | | | (OLFM1), transcript variant 1, | NOELIN; NOELIN1; |
| | | | mRNA. | NOELIN1_V1; |
| | | | | NOELIN1_V2; |
| | | | | NOELIN1_V4 |
| ILMN_1435 | NM_006334.2 | OLFM1 | Homo sapiens olfactomedin 1 | AMY; NOE1; OlfA; |
| | | | (OLFM1), transcript variant 2, | NOELIN; NOELIN1; |
| | | | mRNA. | NOELIN1_V1; |
| | | | | NOELIN1_V2; |
| | | | | NOELIN1_V4 |
| ILMN_21535 | NM_005067.5 | SIAH2 | Homo sapiens seven in absentia | hSiah2 |
| | | | homolog 2 (Drosophila) (SIAH2), | |
| | | | mRNA. | |
| ILMN_4882 | NM_003246.2 | THBS1 | Homo sapiens thrombospondin 1 | TSP; THBS; TSP1 |
| | | | (THBS1), mRNA. | |
| ILMN_18279 | NM_004185.2 | WNT2B | Homo sapiens wingless-type | WNT13; XWNT2 |
| | | | MMTV integration site family, | |
| | | | member 2B (WNT2B), transcript | |
| | | | variant WNT-2B1, mRNA. | |
| ILMN_20088 | NM_001150.1 | ANPEP | Homo sapiens alanyl (membrane) | CD13; LAP1; |
| | | | aminopeptidase (aminopeptidase | PEPN; gp150 |
| | | | N, aminopeptidase M, microsomal | |
| | | | aminopeptidase, CD13, p150) | |
| | | | (ANPEP), mRNA. | |
| ILMN_26976 | NM_138375.1 | CABLES1 | Homo sapiens Cdk5 and Abl | HsT2563; FLJ35924 |
| | | | enzyme substrate 1 (CABLES1), | |
| | | | mRNA. | |
| ILMN_24376 | NM_022783.1 | DEPDC6 | Homo sapiens DEP domain | DEP.6; FLJ12428; |
| | | | containing 6 (DEPDC6), mRNA. | FLJ13854; |
| | | | | DKFZp564B1778 |
| ILMN_17789 | NM_000418.2 | IL4R | Homo sapiens interleukin 4 | CD124; IL4RA |
| | | | receptor (IL4R), transcript variant | |
| | | | 1, mRNA. | |
| ILMN_3945 | NM_182734.1 | PLCB1 | Homo sapiens phospholipase C, | PLC-I; PI-PLC; |
| | | | beta 1 (phosphoinositide-specific) | PLC-154 |
| | | | (PLCB1), transcript variant 2, | |
| | | | mRNA. | |
| ILMN_27252 | NM_002925.3 | RGS10 | Homo sapiens regulator of G- | Impact of Estrogen |
| | | | protein signalling 10 (RGS10), | Receptor on Gene |
| | | | transcript variant 2, mRNA. | Networks Regulated |
| | | | | by Estrogen . . . |
| | | | | Chang 2006 |
| ILMN_7086 | NM_001012661.1 | SLC3A2 | Homo sapiens solute carrier family | 4F2; CD98; MDU1; |
| | | | 3 (activators of dibasic and neutral | 4F2HC; 4T2HC; |
| | | | amino acid transport), member 2 | NACAE; CD98HC |
| | | | (SLC3A2), transcript variant 1, | |
| | | | mRNA. | |
| ILMN_25446 | NM_003486.5 | SLC7A5 | Homo sapiens solute carrier family | E16; CD98; LAT1; |
| | | | 7 (cationic amino acid transporter, | 4F2LC; MPE16; |
| | | | y+ system), member 5 (SLC7A5), | hLAT1; D16S469E |
| | | | mRNA. | |
| ILMN_28750 | NM_000067.1 | CA2 | Homo sapiens carbonic anhydrase | CA II; CA-II |
| | | | II (CA2), mRNA. | |
| ILMN_4674 | NM_005194.2 | CEBPB | Homo sapiens CCAAT/enhancer | LAP; CRP2; TCF5; |
| | | | binding protein (C/EBP), beta | IL6DBP; NF-IL6; |
| | | | (CEBPB), mRNA. | MGC32080; C/EBP- |
| | | | | beta |
| ILMN_6563 | NM_005195.2 | CEBPD | Homo sapiens CCAAT/enhancer | CELF; CRP3; |
| | | | binding protein (C/EBP), delta | C/EBP-delta; NF- |
| | | | (CEBPD), mRNA. | IL6-beta |
| ILMN_13615 | NM_004433.3 | ELF3 | Homo sapiens E74-like factor 3 | ERT; ESX; EPR-1; |
| | | | (ets domain transcription factor, | ESE-1 |
| | | | epithelial-specific) (ELF3), mRNA. | |
| ILMN_7287 | NM_031475.1 | ESPN | Homo sapiens espin (ESPN), | DFNB36; LP2654; |
| | | | mRNA. | DKFZP434A196 |
| ILMN_20831 | NM_001450.3 | FHL2 | Homo sapiens four and a half LIM | DRAL; AAG11; |
| | | | domains 2 (FHL2), transcript | SLIM3 |
| | | | variant 1, mRNA. | |
| ILMN_25543 | NM_014216.3 | ITPK1 | Homo sapiens inositol 1,3,4- | ITRPK1 |
| | | | triphosphate 5/6 kinase (ITPK1), | |
| | | | mRNA. | |
| ILMN_1092 | NM_000228.2 | LAMB3 | Homo sapiens laminin, beta 3 | LAMNB1 |
| | | | (LAMB3), transcript variant 1, | |
| | | | mRNA. | |
| ILMN_20333 | NM_020190.2 | OLFML3 | Homo sapiens olfactomedin-like 3 | OLF44; HNOEL-iso |
| | | | (OLFML3), mRNA. | |
| ILMN_21964 | NM_002648.2 | PIM1 | Homo sapiens pim-1 oncogene | PIM |
| | | | (PIM1), mRNA. | |

TABLE 12

| Name | Accession | Symbol | Definition | Synonym |
|---|---|---|---|---|
| ILMN_4188 | NM_003857.2 | GALR2 | Homo sapiens galanin receptor 2 | GALNR2 |
| | | | (GALR2), mRNA. | |
| ILMN_5566 | NM_000422.1 | KRT17 | Homo sapiens keratin 17 (KRT17), | PC; K17; PC2; |
| | | | mRNA. | PCHC1 |
| ILMN_11289 | NM_002899.2 | RBP1 | Homo sapiens retinol binding | CRBP; RBPC; |
| | | | protein 1, cellular (RBP1), mRNA. | CRBP1; CRABP-I |
| ILMN_12497 | NM_002970.1 | SAT | Homo sapiens | DC21; KFSD; SSAT |
| | | | spermidine/spermine N1- | |
| | | | acetyltransferase (SAT), mRNA. | |
| ILMN_14916 | NM_148973.1 | TNFRSF25 | Homo sapiens tumor necrosis | DR3; TR3; DDR3; |
| | | | factor receptor superfamily, | LARD; APO-3; |
| | | | member 25 (TNFRSF25), | TRAMP; WSL-1; |
| | | | transcript variant 10, mRNA. | WSL-LR; |
| | | | | TNFRSF12 |

1. A method for screening compounds with estrogenic or anti-estrogenic activity comprising the steps of:
(a) providing a cellular system or a sample thereof being capable of expressing at least one gene of Table 1, wherein the system is selected from the group of single cells, cell cultures, tissues, organs and mammals,
(b) incubating at least a portion of the system with compounds to be screened, and
(c) detecting the activity by comparing an expression of the at least one gene of Table 1 in the system with the gene expression in a control cellular system.

2. The method according to claim 1, wherein in step (a) the human Ishikawa plus cell line is provided.

3. The method according to claim 1, wherein in step (c) the gene expression is determined by detecting at least one gene product encoded by the gene(s) of Table 1 and correlating an amount of signal or change in signal with the gene expression in the system.

4. The method according to claim 1, wherein in step (c) the estrogenic activity of a compound is positively detected if the expression involves an up-regulation of genes of Table 2 and/or a down-regulation of genes of Table 3.

5. The method according to claim 1, wherein in step (a) the cellular system or the sample thereof is additionally capable of expressing at least one gene of Table 4, and furthermore in step (c) the expression of at least one gene of Table 4 is compared with the gene expression in the control system.

6. The method according to claim 5, wherein in step (c) the estrogenic activity of a compound is positively detected if the expression involves an up-regulation of genes of Table 5 and/or a down-regulation of genes of Table 6.

7. The method according to claim 1, wherein in step (a) the cellular system or the sample thereof is capable of expressing multiple genes of Table 1 and/or additionally capable of expressing multiple genes of Table 4, and furthermore in step (c) an expression pattern of multiple genes of Table 1 and/or Table 4 is compared with the expression pattern in the control system, thereby characterizing estrogenicity compound-specifically.

8. The method according to claim 7, wherein in step (c) the expression of all genes of Table 7 is compared with the gene expression in the control system, preferably the expression of all genes of Table 1 and Table 4.

9. The method according to claim 7, wherein the expression pattern is determined by a correlation of the multiple genes and/or a magnitude of altered regulation.

10. The method according to claim 1 for screening a therapeutic compound for an estrogen-dependent disease, wherein in step (a) a mammal, preferably a laboratory mammal, is provided, in step (b) the compound to be screened is administered to the mammal, and in step (c) a therapeutic effect is detected via a level of estrogenic or anti-estrogenic activity in a biological sample withdrawn from the mammal in comparison with a mammal showing non-endocrine disrupting and/or endocrine disrupting effects.

11. A method for monitoring physiological and/or pathological conditions, which are caused, mediated and/or propagated by estrogen receptor signaling, wherein an effective amount of at least one compound or a physiologically acceptable salt thereof is administered to a mammal in need of such treatment and an expression of at least one gene of Table 1 is determined in a biological sample withdrawn from the mammal.

12. Use of at least one gene of Table 1 as marker gene for screening compounds with estrogenic or anti-estrogenic activity.

13. Use of multiple genes of Table 1 and optionally Table 4 as marker genes for characterizing estrogenicity compound-specifically.

14. Use of substances specifically interacting with at least one gene product encoded by a gene of Table 1 for detecting estrogenic or anti-estrogenic activity.

15. Kit for use in detection and/or characterization of estrogenic or anti-estrogenic activity comprising substances specifically interacting with at least one gene product encoded by a gene of Table 1.
